(19) United States
(12) Patent Application Publication
(10) Pub. No.: US 2023/0193307 A1
(43) Pub. Date: Jun. 22, 2023

(54) METHODS AND COMPOSITIONS FOR PRODUCTION OF SALINE TOLERANT PLANTS

(71) Applicants: Rory James HORNBY, Kitchener (CA); Luke YOUNG, Kitchener (CA); Agrisea Corporation, San Francisco, CA (US)

(72) Inventors: Rory James HORNBY, Kitchener (CA); Luke YOUNG, Kitchener (CA)

(21) Appl. No.: 17/797,473

(22) PCT Filed: Feb. 5, 2021
(86) PCT No.: PCT/EP2021/052869

§ 371 Date: Aug. 4, 2022

Related U.S. Application Data
(60) Provisional application No. 62/970,605, filed on Feb. 5, 2020.

(51) Int. Cl. C12N 15/82; C07K 14/415; C12N 5/04

(52) U.S. Cl. CPC C12N 15/8273; C07K 14/415; C12N 15/8227; C12N 5/04

(57) ABSTRACT

Described herein are methods, compositions, and systems for production of saline tolerant plants. In some cases, such plants are produced by genome editing.

### BACKGROUND

Soil salinity is one of the most severe problems in agriculture aside from drought. Absorption of excessive salt from saline soils inhibits both root and shoot growth, reduces reproductive activity and affects viability of plants. As a result, salinity is one of the major constraints in geographic range of crop cultivation globally, and, where it does not preclude growth of certain crops nonetheless substantially affects crop productivity. Additionally, salt accumulation as a result of excessive irrigation, improper drainage, or use of reclaimed water places existing agricultural areas at risk, especially as climate change increases irrigation needs in arid/semiarid regions.

### SUMMARY

In some aspects, the present disclosure provides for an engineered plant comprising genome edits such that the plant is configured to have an elevated growth rate in a medium having an ECe of 11 or greater compared to a plant of a same species without the genome modifications, which engineered plant provides a starch content equal to or greater than 56% by weight. In some aspects, the present disclosure provides for an engineered plant comprising genome modifications such that the plant is configured to have an elevated growth rate in a medium having an ECe of 11 or greater compared to a plant of the same species without the genome modifications, which plant is a dicotyledonous angiosperm. In some embodiments, the engineered plant displays an elevated threshold salinity compared to a threshold salinity of a plant of a same species without the genome modifications. In some embodiments, the engineered plant displays a decreased responsiveness to salinity in terms of yield compared to a plant of the same species without the genome modifications, wherein the responsiveness to salinity is measured by a slope of the yield versus salinity. In some embodiments, the engineered plant is configured to have an elevated growth rate in a medium having an ECe of about 15 or greater or an ECe of about 25 or greater, compared to a plant of a same species without the genome modifications. In some embodiments, the plant is an angiosperm. In some embodiments, the plant is a monocotyledonous angiosperm. In some embodiments, the monocotyledonous angiosperm is a cereal crop. In some embodiments, the cereal crop is a maize, rice, soybean, sugar cane, mung bean, quinoa, barley, oat, rye, sorghum, or wheat species. In some embodiments, the cereal crop is rice, soybean, sugar cane and mung bean. In some embodiments, the cereal crop is rice. In some embodiments, the angiosperm is a dicotyledonous angiosperm vegetable crop. In some embodiments, the dicotyledonous angiosperm vegetable crop is a Brassica, Glycine, or Soja genus. In some embodiments, the genome modifications comprise genome modifications to enhance root-specific expression of at least three salinity resistance genes. In some embodiments, the at least three salinity resistance genes comprise: (a) at least one of SOS1 and SOS2; and (b) at least one of NHX1, VHA-A, AHA3, HKT1, SODA1, SODCC1, SOD2, or OSK1. In some embodiments, the at least three salinity resistance genes further comprise VHA-B, P450, PsbO, PsbP, PsbQ, PsbU, PsbV, Delta-1-pyrroline-5-carboxylate synthase 1, or Delta-1-pyrroline-5-carboxylate synthase 2. In some embodiments, the root-specific promoter or enhancer sequences comprise a root hormone-activated promoter or enhancer. In some embodiments, the root hormone is abscisic acid (ABA), ethylene (ETH), gibberellin (GA), or auxin (AUX). In some embodiments, the promoter or enhancer sequences comprise a promoter or enhancer sequence from DREB2A, or an ETH or AUX enhancer sequence. In some embodiments, at least one of the promoter or enhancer sequences comprise at least 6 nucleotides from an enhancer element from DREB2A, or an ETH or AUX enhancer element sequence. In some embodiments, at least one of the promoter or enhancer sequences further comprises a TAF-1, TATA, E2F, G-BOX, or CAAT enhancer sequence. In some embodiments, at least one of the promoter or enhancer sequences is within 150-500 nucleotides of the 5′ end of an open reading frame of the at least three salinity resistance genes. In some embodiments, at least one of the promoter or enhancer sequences comprises a sequence having at least 95% sequence identity to any one of SEQ ID NO: 1-10, wherein the plant cell is from a rice species. In some embodiments, at least one of the promoter or enhancer sequence comprises a sequence having at least 95% sequence identity to any one of SEQ ID NO: 51-60, or a reverse complement thereof. In some embodiments, at least one of the promoter or enhancer sequences comprises at least 10, at least 20, or at least 30 nucleotides.

In some aspects, the present disclosure provides for an engineered plant cell, comprising genome modifications to enhance root-specific expression of at least three salinity resistance genes. In some embodiments, the plant cell comprises at least three insertions of root-specific promoter or enhancer sequences such that the root specific promoter or enhancer sequences are operably linked to the at least three salinity resistance genes. In some embodiments, the at least three salinity resistance genes comprise: (a) at least one of SOS1 and SOS2; and (b) at least one of NHX1, VHA-A, AHA3, HKT1, SODA1, SODCC1, SOD2, or OSK1. In some embodiments, the at least three salinity resistance genes further comprise VHA-B, P450, PsbO, PsbP, PsbQ, PsbU, PsbV, Delta-1-pyrroline-5-carboxylate synthase 1, or Delta-1-pyrroline-5-carboxylate synthase 2. In some embodiments, the root-specific promoter or enhancer sequences comprise a root hormone-activated promoter or enhancer. In some embodiments, the root hormone is abscisic acid (ABA), ethylene (ETH), gibberellin (GA), or auxin (AUX). In some embodiments, the promoter or enhancer sequences comprise a promoter or enhancer sequence from DREB2A, or an ETH or AUX enhancer sequence. In some embodiments, at least one of the promoter or enhancer sequences comprise at least 6 nucleotides from an enhancer element from DREB2A, or an ETH or AUX enhancer element sequence. In some embodiments, at least one of the promoter or enhancer sequences further comprises a TAF-1, TATA, E2F, G-BOX, or CAAT enhancer sequence. In some embodiments, at least one of the promoter or enhancer sequences is within 150-500 nucleotides of the 5′ end of an open reading frame of the at least three salinity resistance genes. In some embodiments, at least one of the promoter or enhancer sequences comprises a sequence having at least 95% sequence identity to any one of SEQ ID NO: 1-10, wherein the plant cell is from a rice species. In some embodiments, at least one of the promoter or enhancer sequence comprises a sequence having at least 95% sequence identity to any one of SEQ ID NO: 51-60, or a reverse complement thereof. In some embodiments, at least one of the promoter or enhancer sequences comprises at least 10, at least 20, or at least 30 nucleotides.

In some aspects, the present disclosure provides for a multicellular structure having modulated salinity tolerance, wherein the multicellular structure comprises one or more plant cells described herein.

In some aspects, the present disclosure provides for a seed comprising a plurality of genome modifications such that the seed is capable of growth in a solution having an ECe of about 11 or greater, which seed is from a plant providing a starch content greater than 56% by weight. The plant can be according to any of the embodiments described herein.

In some aspects, the present disclosure provides for a seed comprising a plurality of genome modifications such that the seed is capable of growth in a solution having an ECe of about 11 or greater, which seed is from a dicotyledonous angiosperm. The plant can be according to any of the embodiments described herein.

In some aspects, the present disclosure provides for a method of improving the salinity tolerance of a multicellular structure comprising a plurality of plant cells, comprising operably linking root-specific promoter or enhancer sequences to at least three salinity resistance genes within genomes of the plurality of plant cells. In some embodiments, the multicellular structure comprises a whole plant, plant tissue, plant organ, plant part, plant reproductive material, or cultured plant tissue. In some embodiments, operably linking root-specific promoter or enhancer sequences to the at least three salinity resistance genes comprises: (a) inducing callus formation from a seed of the plant; (b) biolistically transforming the callus with microcarriers to generate a transformed callus, wherein the microcarriers have adsorbed thereto at least three different DNA sequences comprising: (i) 5′ and 3′ flanking homology arms or adapters corresponding to a 5′ region of each of the at least three salinity resistance genes; and (ii) an internal sequence comprising an enhancer element from DREB2A, or an ETH or AUX enhancer element sequence; and (c) recovering the transformed callus in growth medium to generate the multicellular structure comprising a plurality of plant cells having improved salinity tolerance. In some embodiments, operably linking root-specific promoter or enhancer sequences to the at least three salinity resistance genes comprises introducing a ribonucleoprotein (RNP) into the multicellular structure using electroporation. The method involves (a) inducing callus formation from a seed of said plant; (b) transforming said callus with ribonucleoprotein by electroporation to generate a transformed callus, wherein said ribonucleoprotein includes at least three different DNA sequences comprising: (i) 5′ and 3′ flanking homology arms or adapters corresponding to a 5′ region of each of said at least three salinity resistance genes; and (ii) an internal sequence comprising an enhancer element from DREB2A, or an ETH or AUX enhancer element sequence; and (c) recovering said transformed callus in growth medium to generate said multicellular structure comprising a plurality of plant cells having improved salinity tolerance.

In some embodiments, the at least three DNA sequences comprise at least one promoter or enhancer sequence having at least 95% sequence homology to SEQ ID NO: 1-10 or 19-34 or a reverse complement thereof, wherein the plant is a rice species. In some embodiments, the at least three DNA sequences comprise at least one promoter or enhancer sequence having at least 95% sequence identity to any one of SEQ ID NO: 51-60 or 70-79 or a reverse complement thereof, wherein the plant is a Brassica species. In some embodiments, the microcarriers have adsorbed thereto programmable nucleases with specificity for a 5′ region of the at least three salinity resistance genes. In some embodiments, the programmable nucleases comprise (a) a class II, type II or class II, type V Cas nuclease in complex with guide RNAs directed against a 5′ region of the at least three salinity resistance genes; (b) a transcription activator-like (TAL) effector and nuclease (TALEN) with specificity for a 5′ region of the at least three salinity resistance genes, or (c) a zinc finger nuclease (ZFN) with specificity for a 5′ region of the at least three salinity resistance genes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a diagram of an example yield vs salinity plot, showing the salinity threshold (ECₜ), Yₘₐₓ (Y value corresponding to ECt), and slope (s).

### DETAILED DESCRIPTION

Overview

Increasing irrigation needs due, e.g., to climate change in arid/semiarid regions as well as increasing use of reclaimed water for crop irrigation places existing crop fields at risk to salinization. Additionally, increased food demand has increased interest in reclaiming or colonizing new crop areas previously seen as marginal or inhospitable to food crops. However, natural genetic variation in food crops provides limited opportunity for enhancement of salinity tolerance via crossbreeding strategies. Even relatively saline resistant crops such as rye and barley have threshold salinity values (ECₑs) well below that of saline water sources such as seawater. The limited repertoire of naturally saline tolerant plants also limits the applicability of crossbreeding strategies, as the plant species to be crossbred must generally be in the same genus or closely related genera.

Accordingly, there is need for methods and compositions that improve the saline tolerance of commonly cultivated crops, particularly cereal and vegetable crops cultivated for food. The advent of programmable nucleases such as Cas endonucleases (e.g., Cas9, CpfI), Transcription activator-like effector nucleases (TALENs), and zinc finger nucleases (ZFNs) has improved the ability to make precise genomic edits in plant species; however, the exact genetic number of and identity of genetic edits to achieve a given phenotype (e.g., salinity resistance) are not well-defined.

To meet this need, described herein are methods, compositions, and systems for increasing the salinity tolerance of crops. Multiplex strategies using programmable nucleases to effect multiple genome edits to crop species to enhance salinity tolerance of the crops are provided. In some cases, these genome edits place existing salt tolerance mechanisms found in all plants under a modified root-localized expression profile to effectively manage extremely high salinities, whilst leaving the remaining tissues of the plant to continue typical physiological processes maintaining high yields. Accordingly, in some embodiments, the present disclosure provides for methods and compositions that improve the salinity tolerance of crops without detrimental effects or energy due to, e.g., expression of foreign transgenes or overexpression of salinity resistance factors in unnecessary areas of the crop plant. Additionally, in some embodiments, the genome editing strategies described herein upregulate salt tolerance to enable typical or increased growth in saline conditions comparable with wild-type strains in non-saline conditions.

Definitions

As used herein, the term “medium” generally refers to a natural or man-made substance in solid, semi-solid, or liquid form that can be used to grow a plant. Examples of suitable types of media that can be used in the present disclosure include soil, artificial or non-soil potted mix, water (e.g., for hydroponic growth formats), and agar.

“Salinity,” as used herein, generally refers to a measure of soluble salts in soil or water. “Salt,” as used herein, generally refers to any molecule comprised of a cation, such as sodium (Na⁺), potassium (K⁺), magnesium (Mg²⁺), or calcium(Ca²⁺), and an anion, such as chloride (Cl⁻), bicarbonate (HCO₃⁻), carbonate (CO₃²⁻), or sulfate (SO₄²⁻). Sodium chloride (NaCl) is the most common salt in groundwater and soils. The salinity of soil (or another growth medium) can be expressed: (a) as the salt concentration of the medium in terms of grams per liter (g/L) or (b) in terms of electric conductivity (EC, in deciSiemens per meter—dS/m, or in equivalent units milliMhos per centimeter—mmhos/cm or milliSiemens per centimeter—mS/cm). For soil, salinity can be measured in units of electrical conductivity of a saturated soil paste extract (ECₑ) taken from the root zone of a plant and averaged over time and depth. Soil paste extracts are soil samples that are brought up to their water saturation points (see, e.g., USDA. Diagnosis and improvement of saline and alkali soils. Agriculture Handbook No. 60. (1954), which is incorporated by reference herein). In some embodiments, electrical conductivities are measured on the vacuum-extracted and filtered water extracts from saturated soil paste extracts.

According to the USDA salinity laboratory, “saline” can be defined as a medium having an electrical conductivity of the saturated paste extract (ECe) of 4 dS/m or greater, “slightly saline” (or medium) can be defined as having an electrical conductivity of the saturated paste extract (ECₑ) of between 4 and 8, moderately saline can be defined as having an electrical conductivity of the saturated paste extract (ECₑ) of between 8 and 16, and severely saline can be defined as having an electrical conductivity of the saturated paste extract (ECₑ) of greater than 16; and seawater may have a salt concentration of 30 g/L and an EC of 50 dS/m. However, effects can occur on crops at salinity levels lower than 4 dS/m; so-called sensitive crops can exhibit growth problems at 0.75-1.5 dS/m and many crops can nonetheless experience growth rate decreases at 1.5-3.0 dS/m.

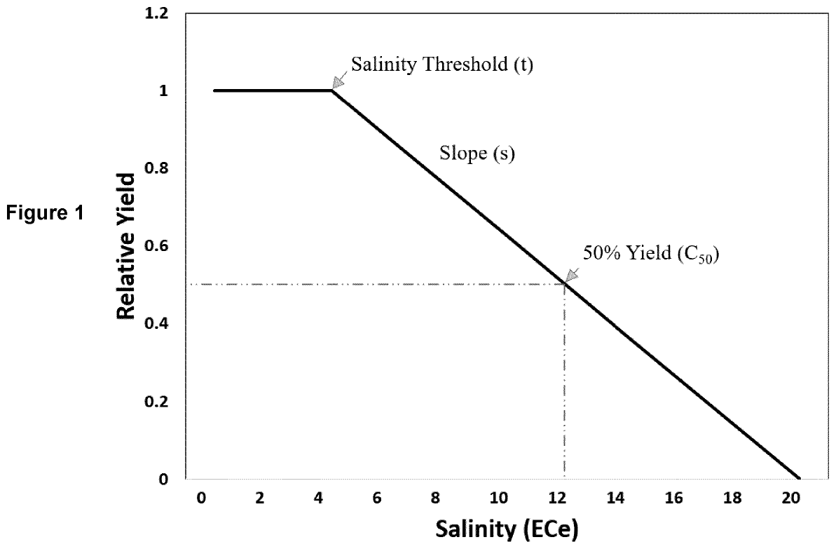

The general effect of salinity on crop plants is to reduce the growth rate resulting in smaller leaves, shorter stature, fewer leaves, and/or decreased yield. In terms of its measurement, crop tolerance to salinity can be described as a function of yield decline across a range of salt concentrations. In some embodiments, crop salt tolerance can be described using two parameters, the threshold (ECₜ), the electrical conductivity that is expected to cause the initial significant reduction in the maximum expected yield (Yₘₐₓ), and the slope (s). FIG. 1 depicts a diagram of an example yield vs salinity plot, showing the salinity threshold (ECₜ), Yₘₐₓ, and slope (s). In terms of ECₜ, plants with ECt of 0.9 dS/m or less can be considered salt sensitive, plants with ECₜ greater than 0.9 up to 1.4 dS/m can be considered moderately sensitive, plants with ECₜ greater than 1.4 up to 2.5 dS/m can be considered moderately tolerant, and plants with ECₜ greater than 2.5 up to 4.0 dS/m can be considered tolerant. In some embodiments, salt tolerance can be expressed in terms of the electrical conductivity of the saturated paste extract (ECₑ) at which yield is reduced by 50% (C₅₀).

As used herein, the term “operatively linked” or “operably linking” generally means that a regulatory element, which can be a synthetic regulatory oligonucleotide of the disclosure or an oligonucleotide to be examined for such activity (e.g., a promoter or enhancer sequence), is positioned with respect to a transcribable or translatable nucleotide sequence such that the regulatory element can affect its regulatory activity. An oligonucleotide having transcriptional enhancer activity, for example, can be located at any distance, including adjacent to or up to thousands of nucleotides away from, and upstream or downstream from the promoter, which can be a minimal promoter element, and nucleotide sequence to be transcribed, and still exert a detectable effect on the level of expression of an encoded reporter molecule.

### Example Embodiments

In some aspects, the present disclosure provides for an engineered plant comprising genome edits such that the plant is configured to have elevated salinity tolerance. Salinity tolerance can be manifested by resistance to individual physicochemical stresses that combine to cause salinity stress such as ionic stress (which can be tested, e.g., by increased resistance to concentrations of LiCl) and/or osmotic stress (which can be tested, e.g., by increased resistance to concentrations of polyethylene glycol). Salinity tolerance can be assessed by reference to yield, mass, length, or growth rate of the whole plant, or by the yield, mass, length, or growth rate of particular parts of the plant (e.g., roots, leaves, shoots, and/or seeds).

In some cases, the engineered plant comprises genome edits such that it has an elevated growth rate in a medium having a particular salt concentration or ECe. The medium may have a salt concentration of at least about 1 gram per liter (g/L), about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L, or about 30 g/L. The medium can have an electrical conductivity of a saturated paste extract (ECₑ) or an electrical conductivity (EC) of at least about 1.7 deciSiemens per meter (dS/m), at least about 2 dS/m, at least about 4 dS/m, at least about 6 dS/m, at least about 8 dS/m, at least about 10 dS/m, at least about 12 dS/m, at least about 14 dS/m, at least about 16 dS/m, at least about 18 dS/m, at least about 20 dS/m, at least about 22 dS/m, at least about 24 dS/m, at least about 26 dS/m, at least about 28 dS/m, at least about 30 dS/m, at least about 32 dS/m, at least about 34 dS/m, at least about 36 dS/m, at least about 38 dS/m, at least about 40 dS/m, at least about 42 dS/m, at least about 44 dS/m, at least about 46 dS/m, at least about 48 dS/m, or at least about 50 dS/m. In some cases, the medium is liquid (e.g., for hydroponic growth strategies). In some cases, the medium is solid (e.g., soil, sand). In some cases, the medium is semi-solid.

In some cases, the engineered plant comprising genome edits such that the plant is configured to have elevated salinity tolerance has a growth rate in a saline medium that is equal to or greater than the growth rate in a non-saline medium. The saline medium can be “slightly saline” (e.g., having an electrical conductivity of the saturated paste extract (ECₑ) or liquid EC of between 4 and 8 dS/m), “moderately saline” (e.g., having an electrical conductivity of the saturated paste extract (ECₑ) or liquid EC of between 8 and 16 dS/m), or “severely saline” can be defined as having an electrical conductivity of the saturated paste extract (ECₑ) or liquid EC of greater than 16 dS/m. The non-saline medium can have an electrical conductivity of the saturated paste extract (ECₑ) or liquid EC of less than 4 dS/m, less than 3 dS/m, less than 2 dS/m, less than 1.7 dS/m, or less than 1.5 dS/m. In some cases the growth rate in saline medium is at least 5%, 10%, 15%, 20%, 25%, 30%, 35%, 40%, 45%, 50%, 100%, 150%, 200% or more.

In some cases, the engineered plant comprising genome edits such that the plant is configured to have elevated salinity tolerance comprises genome edits such that it has a particular threshold salinity (ECO, or an elevated threshold salinity. In some cases, the engineered plant has a threshold salinity of at least about 6 dS/m, at least about 6.5 dS/m, at least about 6.7 dS/m, at least about 7 dS/m, at least about 7.5 dS/m, at least about 8 dS/m, at least about 8.5 dS/m, at least about 9 dS/m, at least about 9.5 dS/m, at least about 10 dS/m, at least about 10.5 dS/m, at least about 11 dS/m, at least about 11.5 dS/m, at least about 12 dS/m, at least about 12.5 dS/m, at least about 13 dS/m, at least about 13.5 dS/m, at least about 14 dS/m, at least about 14.5 dS/m, at least about 15 dS/m, at least about 15.5 dS/m; at least about 16 dS/m, at least about 16.5 dS/m, at least about 17 dS/m, at least about 17.5 dS/m; at least about 18 dS/m, at least about 18.5 dS/m; at least about 19 dS/m, at least about 19.5 dS/m, at least about 20 dS/m, at least about 21 dS/m, at least about 22 dS/m, at least about 23 dS/m at least about 24 dS/m, at least about 25 dS/m, at least about 26 dS/m, at least about 27 dS/m, at least about 28 dS/m, at least about 29 dS/m, or at least about 30 dS/m, or more. In some cases, the elevated threshold salinity is assessed relative to a same plant species or cultivar without the genome edits. In some cases, the threshold salinity is elevated by at least about 1 dS/m, at least about 2 dS/m, at least about 3 dS/m, at least about 4 dS/m, at least about 5 dS/m, at least about 6 dS/m, at least about 7 dS/m, at least about 8 dS/m, at least about 9 dS/m, at least about 10 dS/m, at least about 11 dS/m, at least about 12 dS/m, at least about 13 dS/m, at least about 14 dS/m, or at least about 15 dS/m, or more.

In some cases, the engineered plant comprising genome edits such that the plant is configured to have elevated salinity tolerance comprises genome edits such that it has a particular slope (s) of a yield vs salinity (ECe) plot, or a decreased slope (s) of a yield vs salinity (ECe) plot. In some cases, the decreased slope is assessed relative to a same plant species or cultivar without the genome edits. In some cases, the slope is decreased by at least about 1% per dS/m, at least about 1.5% per dS/m, at least about 2.0% per dS/m, at least about 2.5% per dS/m, at least about 3.0% per dS/m, at least about 3.5% per dS/m, at least about 4.0% per dS/m, at least about 4.5% per dS/m, at least about 5.0% per dS/m, at least about 5.5% per dS/m, at least about 6.0% per dS/m, at least about 8% per dS/m, or at least about 10% per dS/m, or more.

The engineered plant comprising genome edits such that the plant is configured to have elevated salinity tolerance can have a particular starch content as a mature plant, or the seeds or root of the mature plant have can have a particular starch content. In some cases, the plant, root, fruit, or seeds of the mature plant can have at least about 20% starch by weight, at least about 25% starch by weight, at least about 30% starch by weight, at least about 35% starch by weight, at least about 40% starch by weight, at least about 45% starch by weight, at least about 50% starch by weight, at least about 56% starch by weight, at least about 60% starch by weight, at least about 65% starch by weight, at least about 70% starch by weight, at least about 75% starch by weight, or at least about 80% starch by weight, or more. The starch can be amylose or a derivative thereof.

The engineered plant comprising genome edits such that the plant is configured to have elevated salinity tolerance can be an angiosperm. Angiosperms include both monocotyledonous and dicotyledonous angiosperms. Monocotyledonous angiosperms include, but are not limited to, cereal crops, such as maize, rice, sugar cane, barley, millet, oat, rye, sorghum, wheat, or any combination thereof. Dicotyledonous angiosperms include vegetable crops, such as Brassica (e.g. Brassica oleracea, kale), Glycine (e.g. Glycine max), mung bean, quinoa, Soja, or Solanum (e.g. Solanum tuberosum).

The engineered plant comprising genome edits such that the plant is configured to have elevated salinity tolerance can comprise genome modifications to enhance root-specific expression of at least three salinity resistance genes. The genome modifications to enhance root-specific expression of at least three salinity resistance genes can include insertion of promoter or enhancer sequences within the vicinity of the genes according to any of the embodiments described herein.

In some aspects, the present disclosure provides for an engineered plant cell comprising genome modifications to enhance root-specific expression of salinity resistance genes. Enhancement can be accomplished by insertion of a promoter or an enhancer sequence in the vicinity of a salinity resistance gene. In some embodiments, root-specific expression of salinity resistance genes can be accomplished by: (a) insertion of salinity resistance genes under the control of root-tissue-specific (e.g., in rice Os03g01700 and Os02g37190 promoter/enhancer fragments) promoter/enhancer sequences or by operatively linking endogenous salinity resistance genes to root-tissue-specific promoter/enhancer sequences; or by (b) insertion of salinity resistance genes under the control of root-hormone responsive promoter/enhancer sequences or by operatively linking root-hormone responsive promoter/enhancer sequences to endogenous salinity resistance genes.

In the case where root-hormone responsive promoter/enhancer sequences are used, such a strategy may have the advantage of increasing salinity resistance genes in root cells exposed to salinity stresses without the fitness cost of unnecessary expression in non-saline exposed portions of the plant. Example root hormones which promoter/enhancer sequences can be responsive to include abscisic acid (ABA), ethylene (ETH), gibberellin (GA), and auxin (AUX), or any combination thereof. Thus, such promoter and/or enhancer sequences can include DREB2A, ETH, or AUX enhancer/promoter sequences, or any combination thereof. In some cases, such promoter and/or enhancer sequences can include any of the sequences outlined in Table 3, or any combination thereof. The promoter/enhancer sequence can comprise at least 6, at least 7, at least 9, at least 10, at least 11, at least 12, at least 13, at least 14, at least 15, at least 16, at least 17, at least 18, at least 19, or at least 20 nucleotides. The promoter/enhancer sequence can comprise at least 6 nucleotides from DREB2A, ETH, or AUX enhancer/promoter sequences, or from a promoter/enhancer sequence from a root-hormone responsive or root-tissue specific gene.

In some cases, root-hormone responsive promoter/enhancer sequences are modulated by proximity to (e.g., fewer than 15, 14, 13, 12, 11, 10, 9, 8, 7, 6, 5, 4, 3, 2, or 1 nucleotides distance to) a general transcription-enhancing element. Such general transcription-enhancing elements include, but are not limited to, TAF-1, TATA, E2F, G-BOX, or CAAT sequences. The general transcription-enhancing elements can include any of such elements outlined in Table 3, or any combination thereof.

In some cases, root-hormone responsive or root-tissue-specific promoter/enhancer sequences can be inserted in a particular proximity to the 5′ end of an open reading frame of a salinity resistance gene (which can be transgenic or endogenous). The promoter/enhancer sequences can be inserted within at least about 50 nucleotides, at least about 75 nucleotides, at least about 100 nucleotides, at least about 125 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 600 nucleotides, at least about 700 nucleotides, at least about 800 nucleotides, at least about 900 nucleotides, or at least about 1000 nucleotides of the 5′ end of an open reading frame of a salinity resistance gene. The promoter/enhancer sequences can be inserted upstream of the 5′ end of an open reading frame of the salinity resistance gene. The promoter/enhancer sequences can be inserted downstream of the 5′ end of an open reading frame of the salinity resistance gene within an intron.

The salinity resistance genes which the promoters/enhancers are inserted in proximity of can comprise multiple salinity resistance genes. The salinity resistance genes can comprise a plurality of SOS1, SOS2, NHX1, VHA-A, AHA3, HKT1, SODA1, SODCC1, SOD2, and OSK1. The salinity resistance genes can comprise two of SOS1, SOS2, NHX1, VHA-A, AHA3, HKT1, SODA1, SODCC1, SOD2, and OSK1. The salinity resistance genes can comprise three of SOS1, SOS2, NHX1, VHA-A, AHA3, HKT1, SODA1, SODCC1, SOD2, and OSK1. The salinity resistance genes can comprise four of SOS1, SOS2, NHX1, VHA-A, AHA3, HKT1, SODA1, SODCC1, SOD2, and OSK1. The salinity resistance genes can comprise five of SOS1, SOS2, NHX1, VHA-A, AHA3, HKT1, SODA1, SODCC1, SOD2, and OSK1. The salinity resistance genes can comprise six of SOS1, SOS2, NHX1, VHA-A, AHA3, HKT1, SODA1, SODCC1, SOD2, and OSK1. The salinity resistance genes can comprise seven of SOS1, SOS2, NHX1, VHA-A, AHA3, SODA1, SODCC1, SOD2, and OSK1. The salinity resistance genes can comprise eight of SOS1, SOS2, NHX1, VHA-A, AHA3, HKT1, SODA1, SODCC1, SOD2, and OSK1. The salinity resistance genes can comprise nine of SOS1, SOS2, NHX1, VHA-A, AHA3, HKT1, SODA1, SODCC1, SOD2, and OSK1. The salinity resistance genes can comprise all of SOS1, SOS2, NHX1, VHA-A, AHA3, HKT1, SODA1, SODCC1, SOD2, and OSK1. The salinity resistance genes can comprise (a) one of SOS1 and SOS2; and (b) at least one of NHX1, VHA-A, AHA3, HKT1, SODA1, SODCC1, SOD2, and OSK1. The salinity resistance genes can comprise (a) SOS1 and SOS2; and (b) at least one of NHX1, VHA-A, AHA3, HKT1, SODA1, SODCC1, SOD2, and OSK1. The salinity resistance genes can comprise (a) one of SOS1 and SOS2; and (b) at least two of NHX1, VHA-A, AHA3, HKT1, SODA1, SODCC1, SOD2, and OSK1. The salinity resistance genes can comprise (a) one of SOS1 and SOS2; and (b) at least three of NHX1, VHA-A, AHA3, HKT1, SODA1, SODCC1, SOD2, and OSK1. The salinity resistance genes can comprise (a) one of SOS1 and SOS2; and (b) at least four of NHX1, VHA-A, AHA3, HKT1, SODA1, SODCC1, SOD2, and OSK1. The salinity resistance genes can comprise (a) one of SOS1 and SOS2; and (b) at least five of NHX1, VHA-A, AHA3, HKT1, SODA1, SODCC1, SOD2, and OSK1. The salinity resistance genes can comprise (a) one of SOS1 and SOS2; and (b) at least six of NHX1, VHA-A, AHA3, HKT1, SODA1, SODCC1, SOD2, and OSK1. The salinity resistance genes can comprise (a) one of SOS1 and SOS2; and (b) at least seven of NHX1, VHA-A, AHA3, HKT1, SODA1, SODCC1, SOD2, and OSK1. The salinity resistance genes can comprise (a) one of SOS1 and SOS2; and (b) all of NHX1, VHA-A, AHA3, HKT1, SODA1, SODCC1, SOD2, and OSK1. The salinity resistance genes can comprise (a) SOS1 and SOS2; and (b) at least one of NHX1, VHA-A, AHA3, HKT1, SODA1, SODCC1, SOD2, and OSK1. The salinity resistance genes can comprise (a) SOS1 and SOS2; and (b) at least two of NHX1, VHA-A, AHA3, HKT1, SODA1, SODCC1, SOD2, and OSK1. The salinity resistance genes can comprise (a) SOS1 and SOS2; and (b) at least three of NHX1, VHA-A, AHA3, HKT1, SODA1, SODCC1, SOD2, and OSK1. The salinity resistance genes can comprise (a) SOS1 and SOS2; and (b) at least four of NHX1, VHA-A, AHA3, HKT1, SODA1, SODCC1, SOD2, and OSK1. The salinity resistance genes can comprise (a) SOS1 and SOS2; and (b) at least five of NHX1, VHA-A, AHA3, HKT1, SODA1, SODCC1, SOD2, and OSK1. The salinity resistance genes can comprise (a) SOS1 and SOS2; and (b) at least six of NHX1, VHA-A, AHA3, HKT1, SODA1, SODCC1, SOD2, and OSK1. The salinity resistance genes can comprise (a) SOS1 and SOS2; and (b) at least seven of NHX1, VHA-A, AHA3, HKT1, SODA1, SODCC1, SOD2, and OSK1. The salinity resistance genes can comprise any of the named genes in Tables 1 or 4.

In some embodiments, the promoter/enhancer sequences are inserted in the vicinity of a particular locus in the plant genome. In some embodiments, the loci can comprise any of the loci referred to in Tables 1 or 4. In some embodiments, the promoter/enhancer sequences are inserted within at least about 10 nucleotides, at least about 20 nucleotides, at least about 30 nucleotides, at least about 40 nucleotides, at least about 50 nucleotides, at least about 75 nucleotides, at least about 100 nucleotides, at least about 125 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 600 nucleotides, at least about 700 nucleotides, at least about 800 nucleotides, at least about 900 nucleotides, or at least about 1000 nucleotides upstream of the 5′ end of the loci described in Tables 1 or 4.

The salinity resistance genes which the promoters/enhancers are inserted in proximity of can further comprise VHA-B, P450, PsbO, PsbP, PsbQ, PsbU, PsbV, Delta-1-pyrroline-5-carboxylate synthase 1, and Delta-1-pyrroline-5-carboxylate synthase 2. The salinity resistance genes can comprise two of VHA-B, P450, PsbO, PsbP, PsbQ, PsbU, PsbV, Delta-1-pyrroline-5-carboxylate synthase 1, and Delta-1-pyrroline-5-carboxylate synthase 2. The salinity resistance genes can comprise three of VHA-B, P450, PsbO, PsbP, PsbQ, PsbU, PsbV, Delta-1-pyrroline-5-carboxylate synthase 1, and Delta-1-pyrroline-5-carboxylate synthase 2. The salinity resistance genes can comprise four of VHA-B, P450, PsbO, PsbP, PsbQ, PsbU, PsbV, Delta-1-pyrroline-5-carboxylate synthase 1, and Delta-1-pyrroline-5-carboxylate synthase 2. The salinity resistance genes can comprise five of VHA-B, P450, PsbO, PsbP, PsbQ, PsbU, PsbV, Delta-1-pyrroline-5-carboxylate synthase 1, and Delta-1-pyrroline-5-carboxylate synthase 2. The salinity resistance genes can comprise six of VHA-B, P450, PsbO, PsbP, PsbQ, PsbU, PsbV, Delta-1-pyrroline-5-carboxylate synthase 1, and Delta-1-pyrroline-5-carboxylate synthase 2. The salinity resistance genes can comprise seven of VHA-B, P450, PsbO, PsbP, PsbQ, PsbU, PsbV, Delta-1-pyrroline-5-carboxylate synthase 1, and Delta-1-pyrroline-5-carboxylate synthase 2. The salinity resistance genes can comprise eight of VHA-B, P450, PsbO, PsbP, PsbQ, PsbU, PsbV, Delta-1-pyrroline-5-carboxylate synthase 1, and Delta-1-pyrroline-5-carboxylate synthase 2. The salinity resistance genes can comprise all of VHA-B, P450, PsbO, PsbP, PsbQ, PsbU, PsbV, Delta-1-pyrroline-5-carboxylate synthase 1, and Delta-1-pyrroline-5-carboxylate synthase 2.

The root-hormone responsive or root-tissue specific promoter sequence can comprise a particular engineered sequence. The root-hormone responsive or root-tissue specific promoter sequence can comprise a sequence having at least 70% identity to, at least 75% identity to, at least 80% identity to, at least 85% identity to, at least 90% identity to, at least 95% identity to, at least 99% identity to, or a sequence substantially identical to any one of SEQ ID NO: 1-10, when the plant cell is from a rice species.

The root-hormone responsive or root-tissue specific promoter sequence can comprise a particular engineered sequence. The root-hormone responsive or root-tissue specific promoter sequence can comprise a sequence having at least 70% identity to, at least 75% identity to, at least 80% identity to, at least 85% identity to, at least 90% identity to, at least 95% identity to, at least 99% identity to, or a sequence substantially identical to any one of SEQ ID NO: 51-60, when the plant cell is from a Brassica species.

In some aspects, the present disclosure provides for a multicellular structure having modulated salinity tolerance comprising one or more plant cells described herein. The multicellular structure can be a whole plant, plant tissue, plant organ, plant part, plant reproductive material, or cultured plant tissue comprising one or more plant cells described herein. The multicellular structure can be a leaf, a shoot, a seed, a callus, a plantlet, a flower, or an in vitro-cultured bud comprising one or more plant cells described herein. As used herein, the term “callus” is generally intended to include regenerable plant tissue such as embryogenic callus. As used herein, “plantlet” generally includes young or small plants used as propagules. Plantlets may be produced asexually by tissue culture or cell culture. As used herein, “in vitro-cultured bud” generally includes in vitro-propagated apical and axillary buds. Plant apical and axillary buds are small terminal or lateral protuberances on the stem of a vascular plant that may develop into a flower, leaf, or shoot. Plant buds arise from meristem tissue and may consist of overlapping immature leaves or petals. The multicellular structure can be a leaf, a shoot, a seed, a callus, a plantlet, a flower, or an in vitro-cultured bud derived from a plant described herein. The multicellular structure can be a whole plant, plant tissue, plant organ, plant part, plant reproductive material or cultured plant tissue derived from a plant described herein.

In some aspects, the present disclosure provides for a seed having a particular starch concentration comprising a plurality of genome modifications such that the seed is capable of growth in a medium having a salt concentration greater than about 1 gram per liter (g/L), about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L, or about 30 g/L. The medium can have an electrical conductivity of a saturated paste extract (ECe) or an electrical conductivity (EC) of at least about 1.7 deciSiemens per meter (dS/m), at least about 2 dS/m, at least about 4 dS/m, at least about 6 dS/m, at least about 8 dS/m, at least about 10 dS/m, at least about 12 dS/m, at least about 14 dS/m, at least about 16 dS/m, at least about 18 dS/m, at least about 20 dS/m, at least about 22 dS/m, at least about 24 dS/m, at least about 26 dS/m, at least about 28 dS/m, at least about 30 dS/m, at least about 32 dS/m, at least about 34 dS/m, at least about 36 dS/m, at least about 38 dS/m, at least about 40 dS/m, at least about 42 dS/m, at least about 44 dS/m, at least about 46 dS/m, at least about 48 dS/m, or at least about 50 dS/m. In some cases, the medium is liquid (e.g., for hydroponic growth strategies). In some cases, the medium is solid (e.g., soil, sand). In some cases, the medium is semi-solid. In some cases, the starch concentration can be at least about 20% starch by weight, at least about 25% starch by weight, at least about 30% starch by weight, at least about 35% starch by weight, at least about 40% starch by weight, at least about 45% starch by weight, at least about 50% starch by weight, at least about 56% starch by weight, at least about 60% starch by weight, at least about 65% starch by weight, at least about 70% starch by weight, at least about 75% starch by weight, or at least about 80% starch by weight, or more. The starch can be amylose or a derivative thereof.

In some aspects, the present disclosure provides for a dicotyledonous angiosperm seed comprising a plurality of genome modifications such that the seed is capable of growth in a medium having a salt concentration greater than greater than about 1 gram per liter (g/L), about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L, or about 30 g/L. The medium can have an electrical conductivity of a saturated paste extract (ECₑ) or an electrical conductivity (EC) of at least about 1.7 deciSiemens per meter (dS/m), at least about 2 dS/m, at least about 4 dS/m, at least about 6 dS/m, at least about 8 dS/m, at least about 10 dS/m, at least about 12 dS/m, at least about 14 dS/m, at least about 16 dS/m, at least about 18 dS/m, at least about 20 dS/m, at least about 22 dS/m, at least about 24 dS/m, at least about 26 dS/m, at least about 28 dS/m, at least about 30 dS/m, at least about 32 dS/m, at least about 34 dS/m, at least about 36 dS/m, at least about 38 dS/m, at least about 40 dS/m, at least about 42 dS/m, at least about 44 dS/m, at least about 46 dS/m, at least about 48 dS/m, or at least about 50 dS/m. In some cases, the medium is liquid (e.g., for hydroponic growth strategies). In some cases, the medium is solid (e.g., soil, sand). In some cases, the medium is semi-solid. In some cases, the starch concentration can be at least about 20% starch by weight, at least about 25% starch by weight, at least about 30% starch by weight, at least about 35% starch by weight, at least about 40% starch by weight, at least about 45% starch by weight, at least about 50% starch by weight, at least about 56% starch by weight, at least about 60% starch by weight, at least about 65% starch by weight, at least about 70% starch by weight, at least about 75% starch by weight, or at least about 80% starch by weight, or more. The starch can be amylose or a derivative thereof. The dicotyledonous angiosperm can be a vegetable.

In some aspects, the present disclosure provides for a method of improving the salinity tolerance of a multicellular structure comprising a plurality of plant cells comprising operably linking root-specific promoter or enhancer sequences to at least three salinity resistance genes within genomes of the plurality of plant cells. The root specific promoter/enhancer sequence include any of the promoter/enhancer sequences described herein (e.g. any of the sequences described in Tables 1, 2, 3, 4, or 5). The multicellular structure can be a whole plant, plant tissue, plant organ, plant part, plant reproductive material or cultured plant tissue comprising one or more plant cells described herein. The multicellular structure can be a leaf, a shoot, a seed, a callus, a plantlet, a flower, or an in vitro-cultured bud comprising one or more plant cells described herein.

In some embodiments, operably linking root-specific promoter or enhancer sequences to at least three salinity resistance genes comprises a particular transfection procedure. Such a procedure can involve (a) inducing callus formation from a seed of the plant. Such a procedure can then comprise (b) biolistically transforming the callus with microcarriers to generate a transformed callus, wherein the microcarriers have adsorbed thereto at least three different DNA sequences comprising the promoter or enhancer sequences. In some cases, the at least three different DNA sequences can comprise (i) 5′ and 3′ flanking homology arms or adapters corresponding to a 5′ region of each of the at least three salinity resistance genes; an internal sequence comprising a promoter/enhancer element (or at least 5, 6, 7, 8, 9, or 10 nucleotides from an enhancer/promoter element) from DREB2A, or an ETH or AUX promoter/enhancer sequence. Such a procedure can then comprise (c) recovering the transformed callus in growth medium to generate the multicellular structure comprising a plurality of plant cells having improved salinity tolerance.

In some cases, the at least three DNA sequences comprise at least one sequence having at least 70% identity to, at least 75% identity to, at least 80% identity to, at least 85% identity to, at least 90% identity to, at least 95% identity to, at least 99% identity to, or a sequence substantially identical to any one of SEQ ID NO: 1-10 or 19-34 or a reverse complement thereof, wherein the plant is a rice species.

In some cases, the at least three DNA sequences comprise at least one sequence having at least 70% identity to, at least 75% identity to, at least 80% identity to, at least 85% identity to, at least 90% identity to, at least 95% identity to, at least 99% identity to, or a sequence substantially identical to any one of SEQ ID NO: 51-60 or 70-79 or a reverse complement thereof, wherein the plant is a Brassica species.

In some embodiments, the microcarriers have absorbed thereto programmable nucleases with specificity for a 5′ upstream region or an intronic region proximal to the 5′ end of an open reading frame of the at least three salinity resistance genes. The programmable nucleases can comprise a class II, type II or class II, type V Cas nuclease in complex with guide RNAs directed against a 5′ upstream region of the at least three salinity resistance genes or an intronic region proximal to the 5′ end of an open reading frame of the at least three salinity resistance genes. When the programmable nuclease is a Cas nuclease, the guide RNAs can comprise any of the targeting sequences described in Table 1 or Table 4. The programmable nucleases can comprise transcription activator-like (TAL) effector and nucleases (TALENs) with specificity for a 5′ region of the at least three salinity resistance genes or an intronic region proximal to the 5′ end of an open reading frame of the at least three salinity resistance genes. The programmable nucleases can comprise zinc finger nucleases (ZFN) with specificity for a 5′ region of the at least three salinity resistance genes or an intronic region proximal to the 5′ end of an open reading frame of the at least three salinity resistance genes.

In some embodiments, the engineered plants, the engineered plant cells, the multicellular structures and the seeds according to the invention are not produced by a process that involves homologous recombination. In some embodiments, the engineered plants, the engineered plant cells, the multicellular structures and the seeds according to the invention are not produced by an essentially biological process.

In another aspect, the present invention provides a method of producing flour, wholemeal, starch or other product obtained from seed, the method comprising; a) obtaining seed of the invention, and b) extracting the flour, wholemeal, starch or other product.

In another aspect, the present invention provides a method of processing rice, the method comprising; a) obtaining seed of the invention, b) removing the husks, c) milling the shelled rice to remove the bran layer. In some embodiments, the method involves the step of whitening the rice. In some embodiments, the method involves the step of polishing the rice.

TABLE 1
Example Design of Genes/Loci, Targeting Sequences, and Enhancers to be Added for Gene Editing in Rice
(Oryza sativa japonica) According to Some Embodiments Described Herein

| | | | | gRNA | | | |
|---|---|---|---|---|---|---|---|
| | | | | targeting | | SEQ | |
| | | | | sequence | | ID | |
| | | ACCESSION | | (w/PAM | ENHANCER | NO: | |
| | | RICE | | underlined for | ELEMENTS | FOR | ENHANCER |
| GENE | FUNCTION | DATABASE | LOCUS | reference) | INSERTED | ENHANCER | SEQUENCE |
| NHX1 | Vacuolar Na+/H+ | Os07t06669 | chr07: | TTTACCGGGAATTG | DREB2A + GA + | 1 | GCCGACCCT |
| | antiporters | 00-01 | 28,164,424 . . . | TCGAATTAGGC | TAF-1 + | | TTGGCCACG |
| | | | 28,169,307 | (SEQ ID NO: 89) | TATATA | | TGGCAATATATA |
| Os- | Vacuolar H+- | Os06g06620 | chr06: | GTGATCTGACC | TATA + TAF-1 | 2 | GCCGACGCCACGT |
| VHA-A | ATPase | 00 | 27,286,319 . . . | GCCGG | + ETH + | | GGCAAGCCGCCTA |
| | | | 27,292,847 | (SEQ ID NO: 90) | DREB2A | | TATA |
| SOS1 | Na+/H+ | Os12g06411 | chr12: | TTTGCCTTGGATTT | AUX + ETH + | 3 | TGTCTCGCCGCCGCG |
| | antiporter | 00 | 27,495,775 . . . | TCCACTTGCA | E2F + DREB2A | | GGAAA |
| | | | 27,508,468 | (SEQ ID NO: 91) | | | |
| SOS2 | Serine/threonine | Os06g06060 | chr06: | GAGTTGGATTCGTG | AUX + ETH + | 4 | TGTCTCGCCGCCGCG |
| | protein kinase | 00 | 24,038,959 . . . | GCCGCGCGG | E2F + DREB2A | | GGAAAAGCCGACGCG |
| | | | 24,044,785 | (SEQ ID NO: 92) | | | GGAAAA |
| OsAHA | Plasma membrane | Os12g06387 | chr12: | TGGTGGGAGATCCA | AUX + ETH + | 5 | TGTCTCGCCGCCGCG |
| 3 | H+-ATPase pump | 00 | 27,387,215 . . . | TCTGCGAGG | E2F + DREB2A | | GGAAAAGCCGACGCG |
| | out H+ into ECM | | 27,393,762 | (SEQ ID NO: 93) | | | GGAAAA |
| HKT1 | High-affinity K+ | Os06g07017 | chr06: | TTTCTCATACTCGT | DREB2A + E2F- | 6 | GCTCAAGCCGACGCG |
| | transporter | 00 | 29,538,938 . . . | TGGCTCGTTGC | 1 | | GGAAAGGCCGACGCC |
| | | | 29,541,203 | (SEQ ID NO: 94) | | | GCCGCCGAC |
| SODA1 | Manganese | Os05t03239 | chr05: | CCCATCCTCCAGTG | GA + TATA + | 7 | CCTTTGTATATAGCC |
| | superoxide | 00 | 15,046,751- | CTACGG | ETH + DREB2A | | GACGCCGACGCCGCC |
| | dismutase- | | 15,051,399 | (SEQ ID NO: 95) | | | |
| | isolated to the | | | | | | |
| | mitochondria- | | | | | | |
| | converts free | | | | | | |
| | radical | | | | | | |
| | superoxides into | | | | | | |
| | hydrogen | | | | | | |
| | peroxide and | | | | | | |
| | dioxygen | | | | | | |
| SODCC | CuZn superoxide | Os03g03515 | chr03: | TTTAGGACCTCTAG | GA + TATA + | 8 | CCTTTGTATATAGCC |
| 1 | dismutase | 00 | 13,181,396- | AGGCTACTTCTGCC | ETH + DREB2A | | GACGCCGAC |
| | localized to | | 13,184,453 | (SEQ ID NO: 96) | | | |
| | cytoplasm and | | | | | | |
| | ECM | | | | | | |
| OsSOD | CuZn superoxide | Os03g02192 | chr03: | GCGAAGCGGCAGAG | GA + TATA + | 9 | CCTTTGTATATAGCC |
| 2 | dismutase | 00 | 6,271,959- | AATGGCAGGGAAA | ETH + DREB2A | | GACGCCGACGCCGCC |
| | cytoplasm | | 6,275,334 | (SEQ ID NO: 97) | | | |
| OSK1 | Sugar-non- | Os05g05305 | chr05: | TTTGATAATAGCTA | GA + TATA + | 10 | CCTTTGTATATAGCC |
| | fermenting 1- | 00 | 26347148 . . . | TGAAGATTTTGTG | ETH + DREB2A | | GACGCCGAC |
| | related protein | | 26351599 | (SEQ ID NO: 98) | | | |
| | kinase | | | | | | |
| P450 | Leaf Wax | | | | | 11 | |
| | Synthesis Rate | | | | | | |
| | Limiting Enzyme | | | | | | |
| PsbO | Oxygen Evolving | | | | | 12 | |
| | Complex | | | | | | |
| | component in | | | | | | |
| | plants | | | | | | |
| PsbP | Oxygen Evolving | | | | | 13 | |
| | Complex | | | | | | |
| | component in | | | | | | |
| | plants | | | | | | |
| PsbQ | Oxygen Evolving | | | | | 14 | |
| | Complex | | | | | | |
| | component in | | | | | | |
| | plants | | | | | | |
| PsbU | Oxygen Evolving | | | | | 15 | |
| | Complex | | | | | | |
| | component in | | | | | | |
| | cyanobacteria | | | | | | |
| PsbV | Oxygen Evolving | | | | | 16 | |
| | Complex | | | | | | |
| | component in | | | | | | |
| | cyanobacteria | | | | | | |
| Delta | P5CS1. Proline | Os05t04555 | | | | 17 | |
| -1- | biosynthesis | 00-01 | | | | | |
| pyrroline- | leading to | | | | | | |
| 5- | osmoregulation. | | | | | | |
| carboxylate | | | | | | | |
| synthase | | | | | | | |
| 1 | | | | | | | |
| Delta | P5CS2. Proline | Os01t08482 | | | | 18 | |
| -1- | biosynthesis | 00-01 | | | | | |
| pyrroline- | leading to | | | | | | |
| 5- | osmoregulation. | | | | | | |
| carboxylate | | | | | | | |
| synthase | | | | | | | |
| 2 | | | | | | | |

*DNA/RNA sequences are in order from 5′ to 3′

TABLE 2
Example Design of Repair Templates for Insertion of Enhancers in Rice
(Oryza sativa japonica) According to Some Embodiments Described Herein

| | | | | COMPLETE DNA INSERTS | |
|---|---|---|---|---|---|
| | | | | WITH HOMOLOGY ARMS | |
| | | | SEQ | (bold underlining | |
| | | CAS | ID | signifies | |
| | | EN- | NO: | “sticky end” | |
| | | ZYME | FOR | adapters; bold | CAS9 HOMOLOGY |
| | ACCESSION | FOR | DNA | only indicates | ARMS OF DNA |
| | RICE | ED- | IN- | filler nucleotides | INSERT FOR |
| GENE | DATABASE | ITING | SERT | to prevent frameshift) | REFERENCE |
| NHX1 | Os07t0666 | Cpf1 | 19 | GCATGCCGACCCTTTGGCCACGTGGCAAT | N/A |
| | 900-01 | | | ATATATT | |
| | | | 20 | ATCGAATATATATTGCCACGTGGCCAAAG | |
| | | | | GGTCGGC | |
| OS- | Os06g0662 | Cas9 | 21 | CGAGAGAGCCGTCTCCCTCTTCGCTTCTC | CGAGAGAGCCGTCTCCCTCTTCGCT |
| VHA-A | 000 | | | CTCTCCCCCCCGTGATCTGACGCCGACGC | TCTCCTCTCCCCCCCGTGATCTGAC |
| | | | | CACGTGGCAAGCCGCCTATATACGCCGGC | (SEQ ID NO: 99) |
| | | | | GACGACCCCCTCCCACCACCCGCCGCCGC | CGCCGGCGACGACCCCCTCCCACCA |
| | | | | CGCCGCGCCCCCGC | CCCGCCGCCGCCGCCGCGCCCCCGC |
| | | | | | (SEQ ID NO: 100) |
| SOS1 | Os12g064 | Cpf1 | 22 | ACTTAATGTCTCGCCGCCGCGGGAAAAGC | N/A |
| | 1100 | | | CGACGCGGGAAAA | |
| | | | 23 | AAGTTTTTCCCGCGTCGGCTTTTCCCGCG | |
| | | | | GCGGCGAGACATT | |
| SOS2 | Os06g060 | Cas9 | 24 | CGGCGGCGTCGTCGTCTTCCTCCTCTTCC | CGGCGGCGTCGTCGTCTTCCTCCTC |
| | 6000 | | | CCCCGAGTTGGATTCGTGGCCTGTCTCGC | TTCCCCCCGAGTTGGATTCGTGGCC |
| | | | | CGCCGCGGGAAAAGCCGACGCGGGAAAAC | (SEQ ID NO: 101) |
| | | | | GGCGGATGGGAGGGGAGGAGGGAATGGCG | CGGCGGATGGGAGGGGAGGAGGGAA |
| | | | | GCGGGGAGGAAGAAGCGGGT | TGGCGGCGGGGAGGAAGAAGCGGGT |
| | | | | | (SEQ ID NO: 102) |
| OsAHA3 | Os12g063 | Cas9 | 25 | GATACAGGTAGAGAGAGGTGAGAAGGCAG | GATACAGGTAGAGAGAGGTGAGAAG |
| | 8700 | | | TGGTGGGAGATCCATCTTGTCTCGCCGCC | GCAGTGGTGGGAGATCCATCT |
| | | | | GCGGGAAAAGCCGACGCGGGAAAAGCGAG | (SEQ ID NO: 103) |
| | | | | GTGCCTCCATGGCTGAGAAGGAGGGCAAC | GCGAGGTGCCTCCATGGCTGAGAAG |
| | | | | CTCGACGCCGTCCTCAAGGA | GAGGGCAACCTCGACGCCGTCCTCA |
| | | | | | AGGA |
| | | | | | (SEQ ID NO: 104) |
| HKT1 | Os06g070 | Cpf1 | 26 | GCTCAAGCCGACGCGGGAAAGGCCGACGC | N/A |
| | 1700 | | | CGCCGCCGAC | |
| | | | 27 | GAGCGTCGGCGGCGGCGTCGGCCTTTCCC | |
| | | | | GCGTCGGCTT | |
| SODA1 | Os05t032 | Cas9 | 28 | ACCTGCCACTCGCCCACTCCTCCTCCTCC | ACCTGCCACTCGCCCACTCCTCCTC |
| | 3900 | | | CCCATCCTCCAGTGCCTTTGTATATAGCC | CTCCCCCATCCTCCAGTG |
| | | | | GACGCCGACGCCGCCCTACGGTGTCACGC | (SEQ ID NO: 105) |
| | | | | AGCCATGGCGCTCCGCACGCTGGCCTCGA | CTACGGTGTCACGCAGCCATGGCGC |
| | | | | GGAAAACC | TCCGCACGCTGGCCTCGAGGAAAACC |
| | | | | | (SEQ ID NO: 106) |
| SODCC1 | Os03g035 | Cpf1 | 29 | CTGCAACCTTTGTATATAGCCGACGCCGA | N/A |
| | 1500 | | | CGCCGCC | |
| | | | 30 | GCAGGGCGGCGTCGGCGTCGGCTATATAC | |
| | | | | AAAGGTT | |
| OsSOD2 | Os03g02 | Cpf1 | 31 | CCTTTGTATATAGCCGACGCCGACGCCGC | N/A |
| | 19200 | | | CGGGCGA | |
| | | | 32 | ccGGCGGCGTCGGCGTCGGCTATATACAA | |
| | | | | AGGTCGC | |
| OSK1 | Os05g05 | Cpf1 | 33 | TGTGAACCTTTGTATATAGCCGACGCCGA | N/A |
| | 30500 | | | CGCCGCCG | |
| | | | 34 | ACACGGCGGCGTCGGCGTCGGCTATATAC | |
| | | | | AAAGGTT | |
| P450 | | | | | |
| PsbO | | | | | |
| PsbP | | | | | |
| PsbQ | | | | | |
| PsbU | | | | | |
| PsbV | | | | | |
| Delta-1- | Os05t045 | | | | |
| pyrroline- | 5500-01 | | | | |
| 5- | | | | | |
| carboxylate | | | | | |
| synthase | | | | | |
| 1 | | | | | |
| Delta-1- | Os01t084 | | 42 | | |
| pyrroline- | 8200-01 | | | | |
| 5- | | | | | |
| carboxylate | | | | | |
| synthase | | | | | |
| 2 | | | | | |

*DNA/RNA sequences are in order from 5′ to 3′

TABLE 3
Shorthand Code used to refer to enhancer
elements described herein*

| CODE | FULL NAME | RICE | KALE | SEQUENCE |
|---|---|---|---|---|
| DREB | DREB2A-see DREB2A | Y | Y | |
| DREB2A | Oryza sativa | Y | Y | GCCGAC |
| | drought | | | |
| | responsive | | | |
| | element-binding | | | |
| | protein 2A | | | |
| GA | Gibberellin Acid | Y | Y | CCTTTG |
| | Responsive | | | |
| | Element | | | |
| TAF | See TAF-1 | Y | Y | |
| TAF-1 | TAF-1 binding | Y | Y | GCCAC |
| | site | | | GTGGC |
| | | | | (SEQ ID |
| | | | | NO: 107) |
| TATATA | TATA box | Y | Y | TATATA/ |
| | triplicate | | | AATATAT |
| TATA | TATA box | Y | Y | TATA |
| ETH | Ethylene | Y | Y | TAAGAG |
| | Responsive | | | CCGCC |
| | Element | | | (SEQ ID |
| | | | | NO: 108)/ |
| | | | | AGCCGCC |
| AUX | See ARE | Y | Y | |
| ARE | Auxin Responsive | | Y | TGTCTC |
| | Element | | | |
| E2F-1 | E2F Binding Site | Y | Y | GCGGGAAA |
| CAAT | | | Y | GGCCAATC |
| | | | | TCACAAT/ |
| | | | | CCCACT |
| TGA | Auxin inducible | | Y | TGACGTAA |
| | element | | | CTGACG |
| G-BOX | Brassica specific | | Y | ACACGTG |
| | bZIP TF binding | | | (T/GC) |
| | site | | | |
| AUX | D1 Auxin | | Y | CCTCG |
| COMP | Responsive | | | TGTCTC |
| | Element, | | | (SEQ ID |
| | composite element. | | | NO: 109) |

*DNA sequences are in order from 5′ to 3′

TABLE 4
Example Design Genes/Loci, Targeting Sequences, and
Enhancers to be Added for Gene Editing in Kale (Brassica
oleracea) According to some Embodiments Described Herein

| RICE | GENE | | | gRNA targeting | | SEQ | |
|---|---|---|---|---|---|---|---|
| ORTHO- | (ENSEMBL | | | sequence | | ID | |
| LOGUE/ | ACCES- | | | (w/PAM | | NO: | |
| KALE | SION | DESCRIP- | | underlined for | ENHANCERS | FOR | |
| NAME | CODE) | TION | LOCUS | reference) | INSERTED | ENHANCER | ENHANCER SEQUENCE |
| NHX1/Bo | Bo9g0102 | Vacuolar | chrC9: | TTTG TTGTGTAC | CAAT + DREB + | 51 | CACAATGCCGACCCTTT |
| 9g01020 | 00 | Na+/H+ | 2,940,449 | TTGATCGCCGATA | GA + TAF + | | GGCCACGTGGCAATATA |
| 0 | | anti- | - | (SEQ ID NO: | TATATA | | TA |
| | | porter | 2,943,704 | 110) | | | |
| Os-VHA- | Bo6g1224 | H+- | chrC6: | TTTC TCAGGTGG | CAAT + TATA + | 52 | CACAATGCCGACGCCAC |
| A/ | 00 | ATPase | 39,080,50 | TGAATTTTGATCG | TAF-1 + ETH | | GTGGCAAGCCGCCTATA |
| BoVHA-A | | | 8- | (SEQ ID NO: | | | TA |
| | | | 39,084,29 | 111) | | | |
| | | | 6 | | | | |
| SOS1/ | Bo9g0714 | Sodium | chrC9: | TTTC TATACGTA | TGA + CAAT + | 53 | CTGACGCACAATCCCAC |
| BoSOS1 | 70 | proton | 21,527,17 | TACTACTTCCCTC | AUX + ETH + | | TTGTCTCTATATAGCCG |
| | | exchanger; | 5- | (SEQ ID NO: | E2F + DREB + | | CCGCGGGAAAAGCCGAC |
| | | putative | 21,532,99 | 112) | E2F + TATATA | | GCGGGAAAATATATAA |
| | | NHX7 | 7 | | | | |
| | | (SOS1) | | | | | |
| SOS2/ | Bo4g1395 | Serine/ | chrC4: | TTTC CAAATCGC | AUX/ETH + | 54 | CTGACGCACAATCCCAC |
| BoSOS2 | 20.1 | threonine | 37,165,34 | CAGATTATCAGTA | E2F-1 + TATA + | | TTGTCTCTATATAGCCG |
| | | protein | 4- | (SEQ ID NO: | DREB.TATATA | | CCGCGGGAAAAGCCGAC |
| | | kinase | 37,168,14 | 113) | | | GCGGGAAAATATATAA |
| | | | 9 | | | | |
| OsAHA3/ | Bo9g1331 | H+ | chrC9: | TTTC TGAATGAT | AUXCOMP + | 55 | CACAATCCCACTACCTC |
| BoAHA3 | 10 | ATPase | 40,594,33 | TTCTAACGATCAT | AUX/ETH + | | GTGTCTCGCCGCCGCGG |
| | | | 1- | (SEQ ID NO: | E2F-1 + TATA + | | GAAAAGCCGACGCGGGA |
| | | | 40,600,20 | 114) | DREB | | AAATATATA |
| | | | 1 | | | | |
| HKT1/ | Bo3g0446 | Vascular | chrC3: | TTTC ATTATAAT | CAAT + ARE + | 56 | CACAATACCTCGTGTCT |
| BoHKT1 | 60 | sodium | 18,495,56 | TGGGCACATTGTG | DREB2A + E2F-1 | | GCGCCGACGCGGAAAGG |
| | | transporter | 1- | (SEQ ID NO: | | | CCGACGCCGCCGCCGAC |
| | | | 18,499,83 | 115) | | | |
| | | | 9 | | | | |
| [CuZn] | Bo5g1370 | Superoxide | 42,621,36 | TTTC TCGGCCCA | CAAT + GA + | 57 | CACAATCCCACTCCTT |
| SOD2/ | 20 | Dismutase | 6- | TCTACGTGTCATG | TATA + G-BOX + | | GTGTATATAGCCACGC |
| Bo5g137 | | | 42,622,66 | (SEQ ID NO: | DREB2A + AUX | | CGACGCCGCCACCTCG |
| 020 | | | 0 | 116) | | | TGTCTC |
| SODA1/ | Bo1g1413 | Superoxide | chrC1: | TTTA GGCGACCA | CAAT + GA + | 58 | CACAATCCCACTCCTT |
| BoSODA1 | 60 | Dismutase | 40,520,25 | CGTATCTATCTCT | TATA + G-BOX + | | TGTATATAGCCGACGC |
| | | | 6- | (SEQ ID NO: | DREB2A + AUX | | CGACGCCGCCACCTCG |
| | | | 40,521,53 | 117) | | | TGTCTC |
| | | | 6 | | | | |
| CuZn | Bo4g1651 | Superoxide | chrC4: | TTTG CCCAATCG | CAAT + GA + | 59 | CACAATCCCACTCCTT |
| SOD | 50 | dismutase | 44,303,87 | CTTCTTCGAAACG | TATA + G-box + | | TGTATATAGCCGACGC |
| chloro- | | localized | 3- | (SEQ ID NO: | DREB2A + AUX | | CGACGCCGCCACCTCG |
| plast/ | | to | 44,305,48 | 118) | | | TGTCTC |
| CuZn | | chloroplast | 1 | | | | |
| SOD | | and | | | | | |
| chloro- | | stroma | | | | | |
| plast | | | | | | | |
| OSK1/ | Bo4g1380 | SNF-1 | chrC4: | TTTA CTCCTAGT | AUX ENH.AUX + | 60 | CCTCGTGTCTCATGAC |
| Bo4g138 | 00 | related | 36,814,74 | CCTCTACTGTTTT | TGA + CAAT + | | GCACAATCCCACTCCT |
| 000 | | protein | 4- | (SEQ ID NO: | GA + TATA + | | TTGTATATAGCCGACG |
| | | kinase | 36,817,87 | 119) | G-BOX/DREB2A + | | CCGACGCCGCCGTATA |
| | | 1.3 | 9 | | TATA | | TA |
| CuZn | Bo5g0093 | Superoxide | chrC5: | | | 61 | |
| SOD | 10 | dismutase | 3,385,085 | | | | |
| nucleus/ | | localized | - | | | | |
| Bo5g009 | | to | 3,386,559 | | | | |
| 310 | | nucleus | | | | | |
| | | and | | | | | |
| | | cytoplasm. | | | | | |
| P450 | | Leaf | | | | 62 | |
| | | Wax | | | | | |
| | | Synthesis | | | | | |
| | | Rate | | | | | |
| | | Limiting | | | | | |
| | | Enzyme | | | | | |
| PsbO | | Oxygen | | | | 63 | |
| | | Evolving | | | | | |
| | | Complex | | | | | |
| | | component | | | | | |
| | | in | | | | | |
| | | plants | | | | | |
| PsbP | | Oxygen | | | | 64 | |
| | | Evolving | | | | | |
| | | Complex | | | | | |
| | | component | | | | | |
| | | in | | | | | |
| | | plants | | | | | |
| PsbQ | | Oxygen | | | | 65 | |
| | | Evolving | | | | | |
| | | Complex | | | | | |
| | | component | | | | | |
| | | in | | | | | |
| | | plants | | | | | |
| PsbU | | Oxygen | | | | 66 | |
| | | Evolving | | | | | |
| | | Complex | | | | | |
| | | component | | | | | |
| | | in | | | | | |
| | | cyanobacteria | | | | | |
| PsbV | | Oxygen | | | | 67 | |
| | | Evolving | | | | | |
| | | Complex | | | | | |
| | | component | | | | | |
| | | in | | | | | |
| | | cyanobacteria | | | | | |
| P5CS1 | | Delta- | | | | 68 | |
| | | 1- | | | | | |
| | | pyrroline- | | | | | |
| | | 5- | | | | | |
| | | carboxylate | | | | | |
| | | synthase | | | | | |
| | | 1 | | | | | |
| P5CS2 | | Delta- | | | | 69 | |
| | | 1- | | | | | |
| | | pyrroline- | | | | | |
| | | 5- | | | | | |
| | | carboxylate | | | | | |
| | | synthase | | | | | |
| | | 2 | | | | | |

*DNA/RNA sequences are in order from 5′ to 3′

TABLE 5
Example Design of Repair Templates for Insertion of Enhancers in
Kale (Brassica oleracea) According to Some
Embodiments Described Herein*

| | | RE- | | SEQ | | |
|---|---|---|---|---|---|---|
| | | STRIC- | | ID | Cpf1 DNA INSERT | Cpf1 DNA INSERT |
| | | TION | | NO: | FORWARD (bold | REVERSE (bold |
| | | ENZYMES | | FOR | underlining | underlining |
| | | TO | | FOR- | signifies | signifies adapter |
| RICE | | CUT | CAS | WARD | adapter sequences | sequences |
| ORTHO- | | DNA | EN- | DNA | cleaved by | cleaved by |
| LOGUE | GENE | INSERT | ZYME | INSERT | restriction enzymes) | restriction enzymes) |
| NHX1 | Bo9g010 | NdeIII | Cpf1 | 70 | ATAGCACAATGCCGACCCTTTGGCCACGTG | CTATTATATATTGCCACGTGGCCAAAGGGTCGG |
| | 200 | | | | GCAATATATA | CATTGTG (SEQ ID NO: 120) |
| Os- | Bo6g122 | None | Cpf1 | 71 | TCGGCACAATGCCGACGCCACGTGGCAAGC | CCGATATATAGGCGGCTTGCCACGTGGCGTCGG |
| VHA-A | 400 | | | | CGCCTATATA | CATTGTG (SEQ ID NO: 121) |
| SOS1 | Bo9g071 | MnII | Cpf1 | 72 | CTCTTGACGCACAATCCCACTTGTCTCTAT | AGAGTTATATATTTTCCCGCGTCGGCTTTTCCC |
| | 470 | | | | ATAGCCGCCGCGGGAAAAGCCGACGCGGGA | GCGGCGGCTATATAGAGACAAGTGGGATTGTGC |
| | | | | | AAATATATAA | GTCA (SEQ ID NO: 122) |
| SOS2 | Bo4g139 | None | Cpf1 | 73 | GTAACTGACGCACAATCCCACTTGTCTCTA | TTACGCTTATATATTTTCCCGCGTCGGCTTTTC |
| | 520.1 | | | | TATAGCCGCCGCGGGAAAAGCCGACGCGGG | CCGCGGCGGCTATATAGAGACAAGTGGGATTGT |
| | | | | | AAAATATATAAGC | GCGTCAG (SEQ ID NO: 123) |
| OsAHA3 | Bo9g133 | NdeII | Cpf1 | 74 | CATCCACAATCCCACTACCTCGTGTCTCGC | GATGTATATATTTTCCCGCGTCGGCTTTTCCCG |
| | 110 | | | | CGCCGCGGGAAAAGCCGACGCGGGAAAATA | CGGCGGCGAGACACGAGGTAGTGGGATTGTG |
| | | | | | TATA | (SEQ ID NO: 124) |
| HKT1 | Bo3g044 | Bsp | Cpf1 | 75 | ACACCACAATACCTCGTGTCTCGCCGACGC | GTGTGTCGGCGGCGGCGTCGGCCTTTCCCGCGT |
| | 660 | 1286I, | | | GGGAAAGGCCGACGCCGCCGCCGAC | CGGCGAGACACGAGGTATTGTG |
| | | BaeGI, | | | | (SEQ ID NO: 125) |
| | | DRIII | | | | |
| [CuZn] | Bo5g137 | SmiMI, | Cpf1 | 76 | ATGACACAATCCCACTCCTTTGTATATAGC | TCATGAGACACGAGGTGGCGGCGTCGGCGTCGG |
| SOD2 | 020 | NlaIII, | | | CGACGCCGACGCCGCCACCTCGTGTCTC | CTATATACAAAGGAGTGGGATTGTG |
| | | MaeII, | | | | (SEQ ID NO: 126) |
| | | Ppu21l | | | | |
| | | AfIII | | | | |
| SODA1 | Bo1g141 | None | Cpf1 | 77 | TCTCCACAATCCCACTCCTTTGTATATAGC | GAGAGAGACACGAGGTGGCGGCGTCGGCGTCGG |
| | 360 | | | | CGACGCCGACGCCGCCACCTCGTGTCTC | CTATATACAAAGGAGTGGGATTGTG |
| | | | | | | (SEQ ID NO: 127) |
| CuZn SOD | Bo4g165 | NspV; | Cpf1 | 78 | ACGCCACAATCCCACTCCTTTGTATATAGC | GCGTGAGACACGAGGTGGCGGCGTCGGCGTCGG |
| chloro- | 150 | TaqI | | | CGACGCCGACGCCGCCACCTCGTGTCTC | CTATATACAAAGGAGTGGGATTGTG |
| plast | | | | | | (SEQ ID NO: 128) |
| OSK1 | Bo4g138 | HpyCH4 | Cpf1 | 79 | TTTCCCTCGTGTCTCATGACGCACAATCCC | GAAATATATACGGCGGCGTCGGCGTCGGCTATA |
| | 000 | III | | | ACTCCTTTGTATATAGCCGACGCCGACGCC | TACAAAGGAGTGGGATTGTGCGTCATGAGACAC |
| | | | | | GCCGTATATA | GAGG (SEQ ID NO: 129) |
| CuZn SOD | Bo5g009 | None | | 80 | | |
| nucleus | 310 | | | | | |
| P450 | | | | 81 | | |
| PsbO | | | | 82 | | |
| PsbP | | | | 83 | | |
| PsbQ | | | | 84 | | |
| PsbU | | | | 85 | | |
| PsbV | | | | 86 | | |
| P5CS1 | | | | 87 | | |
| P5CS2 | | | | 88 | | |

*DNA sequences are in order from 5′ to 3′

### EXAMPLES

### Example 1. —Transformation of Plants Using DNA Molecules Described Herein for Gene Editing

Plants described herein can be generated e.g., via particle bombardment using a gene gun system. After generating embryogenic callus tissue from either seeds or explants, stem cells are transferred onto Osmotic Media for four hours prior to transformation, whilst remaining in dark conditions. The Osmotic Media contains 4.43 g of Murashige & Skoog Basal Salt, 30 g Sucrose, 90 g Mannitol, and 5 mg 2,4-Dichlorophenoxyacetic acid. During this incubation period the genetic material is prepared. For particle bombardment DNA is precipitated onto gold particles (1 μm in diameter). DNA-coated gold particles are generated by mixing e.g. 120 μl of 40 mg ml⁻¹ gold particles, 5 μl of 1 μg μl⁻¹ Cpfl/Cas9/crRNA (or programmable nuclease) mix, 5 μl of 1 μg μl⁻¹ dsDNA inserts, 40 μl Spermidine, and 100 μl of 2.5M CaCl₂. DNA coated particles are centrifuged, the supernatant is removed and replaced with 75% ethanol, and the gold particles are re-suspended. After this incubation period, calluses on Osmotic Media are moved into the gene gun system, where under a vacuum pressure of at least −5 Hg, 10 μl of prepared DNA-coated gold particles are fired at least 1100 psi. Particles enter the stem cells and the DNA is integrated into the nucleus, resulting in successful transformations; recovery in standard growth media generates the transformed plant or plant cells.

### Example 2.—Generation of Embryogenic Callus Tissue for Particle Bombardment

Seeds were de-hulled by hand or using tweezers. The surface of the seeds was then sterilized with 75% Ethanol for 1 min, followed by 2.5% Sodium Hypochlorite for 15 mins and then washed with distilled water 8 times. The seeds husks were then autoclaved.

The seeds were then placed on Rice Callus Induction Media (4.3g L⁻¹ Murashige & Skoog Basal Salts and Vitamins, 30 g L⁻¹ Maltose, 0.3 g L⁻¹ Casein Hydrolysate, 0.6 g L⁻¹L-Proline, 3 mg L⁻¹ 2,4-Dichlorophenoxyacetic acid (2,4-D), 0.25 mg L⁻¹ 6-Benzylaminopurine (BAP), 3 g L⁻¹ Phytagel adjusted to pH 5.8, autoclaved and the 50 mg L⁻¹/90 mg L⁻¹ Kanamycin/Cefotaxime and 20 mg L⁻¹ Carbendazim was added). The seeds were then incubated in the dark for 14 days (24 h dark@25° C.). This produces embryogenic calli which look white or yellowish and have a compact & nodular structure.

After 14 days the embryogenic calli were cut from the seed and further split into equal segments no larger than 5 mm in diameter. These calli were transferred onto fresh on Rice Callus Induction Media and incubated in the same conditions (24 h dark@25° C.) for 4 days.

### Example 3.—Recovery of Transformed Plants After Particle Bombardment

After bombardment the calli were placed on Rice Osmotic Medium at 26° C. in the dark for 16 hr-20 hr.

Rice Osmotic Media (ROM)

| | | |
|---|---|---|
| 4.3 | g L⁻¹ | Murashige & Skoog Basal Salts and Vitamins |
| 30 | g L⁻¹ | Maltose |
| 90 | g L⁻¹ | Mannitol |
| 0.3 | g L⁻¹ | Casein Hydrolysate |
| 0.6 | g L⁻¹ | L-Proline |
| 3 | mg L⁻¹ | 2,4-Dichlorophenoxyacetic acid (2,4-D) |
| 0.25 | mg L⁻¹ | 6-Benzylaminopurine (BAP) |
| 3 | g L⁻¹ | Phytagel |

Adjust pH to 5.8
Autoclave

| 50 mg L⁻¹/90 mg L⁻¹ | | Kanamycin/Cefotaxime (either antibiotic) |
|---|---|---|
| 20 | mg L⁻¹ | Carbendazim (antifungal) |

The calli were then transferred to Rice Callus Induction Media and incubated in the dark for 7-14 days at 25° C. The calli were then transferred to Rice Regeneration Media I (RRM I) and incubated under light conditions (16 h light:8 h dark at 25° C.) for one to three weeks.

Rice Regeneration Media I (RRM I)

| | | |
|---|---|---|
| 4.3 | g L⁻¹ | Murashige & Skoog Basal Salts |
| 30 | g L⁻¹ | Maltose |
| 2 | g L⁻¹ | Casein Hydrolysate |
| 30 | g L⁻¹ | Sorbitol |
| 0.5 | mg L⁻¹ | Naphthaleneacetic acid (NAA) |
| 1.5 | mg L⁻¹ | Kinetin |
| 0.5 | mg L⁻¹ | BAP |
| 0.5 | mg L⁻¹ | TDZ |
| 4 | g L⁻¹ | Phytagel |

Adjust pH to 5.8
Autoclave

| | | |
|---|---|---|
| 50 | mg L⁻¹ | Cefotaxime or Vancomycin |
| 20 | mg L⁻¹ | Carbendazim (antifungal) |

The resulting plantlets were then transferred onto Rice Regeneration Media II (RRM II).

Rice Regeneration Media II (RRM II)

| | | |
|---|---|---|
| 4.3 | g L⁻¹ | Murashige & Skoog Basal Salts |
| 30 | g L⁻¹ | Maltose (note don't add sugar for growth |
| | | in hydroponics or with bacteria) |
| 100 | mg L⁻¹ | Myo-inositol |
| 60 | mg L⁻¹ | Calcium Silicate |
| 3 | g L⁻¹ | Gelzan |

Adjust pH to 5.8
Autoclave

| | | |
|---|---|---|
| 50 | mg L⁻¹ | Cefotaxime or Vancomycin |
| 20 | mg L⁻¹ | Carbendazim (antifungal) |

### Example 4.—Salinity Testing

The salt tolerance of the transformed plant can be tested by partially submerging the roots of the plantlets or a plant in Hydroponics Media initially with 0 g of salt.

Hydroponics Media (HM)

| | | |
|---|---|---|
| 4.3 | g L⁻¹ | Murashige & Skoog Basal Salts and Vitamins |
| 100 | mg L⁻¹ | Myo-inositol |
| 60 | mg L⁻¹ | Calcium Silicate |

Adjust pH to 5.8
Autoclave

| 50 mg L⁻¹/90 mg L⁻¹ | | Kanamycin/Cefotaxime (either antibiotic) |
|---|---|---|
| 20 | mg L⁻¹ | Carbendazim (antifungal) |

The salinity of the Hydroponics Media is then increased incrementally up to 35 g L⁻¹, which is full oceanic salinity. An engineered plant according to the invention is able to reach the flowing stage of the plant's life cycle while exposed to full oceanic salinity.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-12. (canceled)

13. An engineered plant or plant cell, comprising genome modifications to enhance root-specific expression of at least three salinity resistance genes.

14. The engineered plant or plant cell of claim 13, wherein said plant cell comprises at least three insertions of root-specific promoter or enhancer sequences such that said root specific promoter or enhancer sequences are operably linked to said at least three salinity resistance genes.

15. The engineered plant or plant cell of claim 13, wherein said at least three salinity resistance genes comprise: (a) at least one of SOS1 and SOS2; and (b) at least one of NHX1, VHA-A, AHA3, HKT1, SODA1, SODCC1, SOD2, or OSK1, optionally wherein said at least three salinity resistance genes further comprise VHA-B, P450, PsbO, PsbP, PsbQ, PsbU, PsbV, Delta-1-pyrroline-5-carboxylate synthase 1, or Delta-1-pyrroline-5-carboxylate synthase 2.

16. (canceled)

17. The engineered plant or plant cell of claim 13, wherein said root-specific promoter or enhancer sequences comprise a root hormone-activated promoter or enhancer, optionally wherein said root hormone is abscisic acid (ABA), ethylene (ETH), gibberellin (GA), or auxin (AUX), further optionally wherein said promoter or enhancer sequences comprise a promoter or enhancer sequence from DREB2A, or an ETH or AUX enhancer sequence.

18-19. (canceled)

20. The engineered plant or plant cell of claim 17, wherein at least one of said promoter or enhancer sequences comprise at least 6 nucleotides from an enhancer element from DREB2A, or an ETH or AUX enhancer element sequence, optionally wherein at least one of said promoter or enhancer sequences further comprises a TAF-1, TATA, E2F, G-BOX, or CAAT enhancer sequence.

21. (canceled)

22. The engineered plant or plant cell of claim 17, wherein at least one of said promoter or enhancer sequences is within 50-500 nucleotides of the 5′ end of an open reading frame of said at least three salinity resistance genes.

23. The engineered plant or plant cell of claim 17, wherein at least one of said promoter or enhancer sequences comprises a sequence having at least 95% sequence identity to any one of SEQ ID NO: 1-10, wherein said plant cell is from a rice species.

24. The engineered plant or plant cell of claim 17, wherein at least one of said promoter or enhancer sequence comprises a sequence having at least 95% sequence identity to any one of SEQ ID NO: 51-60, or a reverse complement thereof.

25. The engineered plant or plant cell of claim 17, wherein at least one of said promoter or enhancer sequences comprises at least 10, at least 20, or at least 30 nucleotides.

26-28. (canceled)

29. The engineered plant or plant cell according to claim 1, wherein the engineered plant or plant cell was not produced by a process that involves homologous recombination or was not produced by an essentially biological process.

30. (canceled)

31. A method of improving the salinity tolerance of a multicellular structure comprising a plurality of plant cells, comprising operably linking root-specific promoter or enhancer sequences to at least three salinity resistance genes within genomes of said plurality of plant cells.

32. The method of claim 31 wherein said multicellular structure comprises a whole plant, plant tissue, plant organ, plant part, plant reproductive material, or cultured plant tissue.

33. The method of claim 31, wherein operably linking root-specific promoter or enhancer sequences to said at least three salinity resistance genes comprises:
(a) inducing callus formation from a seed of said plant;
(b) biolistically transforming said callus with microcarriers to generate a transformed callus, wherein said microcarriers have adsorbed thereto at least three different DNA sequences comprising:
(i) 5′ and 3′ flanking homology arms or adapters corresponding to a 5′ region of each of said at least three salinity resistance genes; and
(ii) an internal sequence comprising an enhancer element from DREB2A, or an ETH or AUX enhancer element sequence; and
(c) recovering said transformed callus in growth medium to generate said multicellular structure comprising a plurality of plant cells having improved salinity tolerance.

34. The method of claim 31, wherein:
(a) said at least three DNA sequences comprise at least one promoter or enhancer sequence having at least 95% sequence homology to SEQ ID NO: 1-10 or 19-34 or a reverse complement thereof, wherein said plant is a rice species; or said at least three DNA sequences comprise at least one promoter or enhancer sequence having at least 95% sequence identity to any one of SEQ ID NO: 51-60 or 70-79 or a reverse complement thereof, wherein said plant is a Brassica species.

35. (canceled)

36. The method of claim 31, wherein said microcarriers have adsorbed thereto programmable nucleases with specificity for a 5′ region of said at least three salinity resistance genes, optionally wherein said programmable nucleases comprise
(a) a class II, type II or class II, type V Cas nuclease in complex with guide RNAs directed against a 5′ region of said at least three salinity resistance genes;
(b) transcription activator-like (TAL) effector and nucleases (TALENs) with specificity for a 5′ region of said at least three salinity resistance genes, or
(c) zinc finger nucleases (ZFN) with specificity for a 5′ region of said at least three salinity resistance genes.

37. (canceled)

38. The engineered plant or plant cell of claim 13, wherein said plant is an angiosperm, optionally wherein said plant is a monocotyledonous angiosperm or dicotyledonous angiosperm vegetable crop.

39. The engineered plant or plant cell of claim 38, wherein:
(a) said monocotyledonous angiosperm is a cereal crop, optionally wherein said cereal crop is a maize, rice, barley, oat, rye, sorghum, or wheat species; or
(b) said dicotyledonous angiosperm vegetable crop is a Brassica, Glycine, or Soja genus.

40. The engineered plant or plant cell of claim 13, wherein:
(a) said engineered plant displays an elevated threshold salinity compared to a threshold salinity of a plant of a same species without said genome modifications; or
(b) said engineered plant displays a decreased responsiveness to salinity in terms of yield compared to a plant of the same species without said genome modifications, wherein said responsiveness to salinity is measured by a slope of said yield versus salinity; or
(c) said engineered plant is configured to have an elevated growth rate in a medium having an ECe of about 15 or greater or an ECe of about 25 or greater, compared to a plant of a same species without said genome modifications.

41. A plant part of the engineered plant according to claim 13.

42. The plant part according to claim 41, wherein the plant part is a seed, a leaf, a shoot, a stem, a fruit, or a root.


SEQUENCE LISTING (129 sequences)

SEQ ID NO: 1 - DNA, 30 bp, Artificial Sequence
  Synthetic Construct
gccgaccctttggccacgtggcaatatata

SEQ ID NO: 2 - DNA, 30 bp, Artificial Sequence
  Synthetic Construct
gccgacgccacgtggcaagccgcctatata

SEQ ID NO: 3 - DNA, 20 bp, Artificial Sequence
  Synthetic Construct
tgtctcgccgccgcgggaaa

SEQ ID NO: 4 - DNA, 36 bp, Artificial Sequence
  Synthetic Construct
tgtctcgccgccgcgggaaaagccgacgcgggaaaa

SEQ ID NO: 5 - DNA, 36 bp, Artificial Sequence; identical to SEQ ID NO: 4
  Synthetic Construct

SEQ ID NO: 6 - DNA, 39 bp, Artificial Sequence
  Synthetic Construct
gctcaagccgacgcgggaaaggccgacgccgccgccgac

SEQ ID NO: 7 - DNA, 30 bp, Artificial Sequence
  Synthetic Construct
cctttgtatatagccgacgccgacgccgcc

SEQ ID NO: 8 - DNA, 24 bp, Artificial Sequence
  Synthetic Construct
cctttgtatatagccgacgccgac

SEQ ID NO: 9 - DNA, 30 bp, Artificial Sequence; identical to SEQ ID NO: 7
  Synthetic Construct

SEQ ID NO: 10 - DNA, 24 bp, Artificial Sequence
  Synthetic Construct
cctttgtatatagccgacgccgac

SEQ ID NO: 11-18 - skipped (8 entries)

SEQ ID NO: 19 - DNA, 36 bp, Artificial Sequence
  Synthetic Construct
gcatgccgaccctttggccacgtggcaatatatatt

SEQ ID NO: 20 - DNA, 36 bp, Artificial Sequence
  Synthetic Construct
atcgaatatatattgccacgtggccaaagggtcggc

SEQ ID NO: 21 - DNA, 130 bp, Artificial Sequence
  Synthetic Construct
cgagagagccgtctccctcttcgcttctcctctcccccccgtgatctgacgccgacgccacgtggcaagccgcctatatacgccggcgacgaccccctcccaccacccgccgccgccgccgcgcccccgc

SEQ ID NO: 22 - DNA, 42 bp, Artificial Sequence
  Synthetic Construct
acttaatgtctcgccgccgcgggaaaagccgacgcgggaaaa

SEQ ID NO: 23 - DNA, 42 bp, Artificial Sequence
  Synthetic Construct
aagtttttcccgcgtcggcttttcccgcggcggcgagacatt

SEQ ID NO: 24 - DNA, 136 bp, Artificial Sequence
  Synthetic Construct
cggcggcgtcgtcgtcttcctcctcttccccccgagttggattcgtggcctgtctcgccgccgcgggaaaagccgacgcgggaaaacggcggatgggaggggaggagggaatggcggcggggaggaagaagcgggt

SEQ ID NO: 25 - DNA, 136 bp, Artificial Sequence
  Synthetic Construct
gatacaggtagagagaggtgagaaggcagtggtgggagatccatcttgtctcgccgccgcgggaaaagccgacgcgggaaaagcgaggtgcctccatggctgagaaggagggcaacctcgacgccgtcctcaagga

SEQ ID NO: 26 - DNA, 39 bp, Artificial Sequence; identical to SEQ ID NO: 6
  Synthetic Construct

SEQ ID NO: 27 - DNA, 39 bp, Artificial Sequence
  Synthetic Construct
gagcgtcggcggcggcgtcggcctttcccgcgtcggctt

SEQ ID NO: 28 - DNA, 124 bp, Artificial Sequence
  Synthetic Construct
acctgccactcgcccactcctcctcctcccccatcctccagtgcctttgtatatagccgacgccgacgccgccctacggtgtcacgcagccatggcgctccgcacgctggcctcgaggaaaacc

SEQ ID NO: 29 - DNA, 36 bp, Artificial Sequence
  Synthetic Construct
ctgcaacctttgtatatagccgacgccgacgccgcc

SEQ ID NO: 30 - DNA, 36 bp, Artificial Sequence
  Synthetic Construct
gcagggcggcgtcggcgtcggctatatacaaaggtt

SEQ ID NO: 31 - DNA, 36 bp, Artificial Sequence
  Synthetic Construct
cctttgtatatagccgacgccgacgccgccgggcga

SEQ ID NO: 32 - DNA, 36 bp, Artificial Sequence
  Synthetic Construct
ccggcggcgtcggcgtcggctatatacaaaggtcgc

SEQ ID NO: 33 - DNA, 37 bp, Artificial Sequence
  Synthetic Construct
tgtgaacctttgtatatagccgacgccgacgccgccg

SEQ ID NO: 34 - DNA, 36 bp, Artificial Sequence
  Synthetic Construct
acacggcggcgtcggcgtcggctatatacaaaggtt

SEQ ID NO: 35-50 - skipped (16 entries)

SEQ ID NO: 51 - DNA, 36 bp, Artificial Sequence
  Synthetic Construct
cacaatgccgaccctttggccacgtggcaatatata

SEQ ID NO: 52 - DNA, 36 bp, Artificial Sequence
  Synthetic Construct
cacaatgccgacgccacgtggcaagccgcctatata

SEQ ID NO: 53 - DNA, 67 bp, Artificial Sequence
  Synthetic Construct
ctgacgcacaatcccacttgtctctatatagccgccgcgggaaaagccgacgcgggaaaatatataa

SEQ ID NO: 54 - DNA, 67 bp, Artificial Sequence; identical to SEQ ID NO: 53
  Synthetic Construct

SEQ ID NO: 55 - DNA, 60 bp, Artificial Sequence
  Synthetic Construct
cacaatcccactacctcgtgtctcgccgccgcgggaaaagccgacgcgggaaaatatata

SEQ ID NO: 56 - DNA, 51 bp, Artificial Sequence
  Synthetic Construct
cacaatacctcgtgtctcgccgacgcgggaaaggccgacgccgccgccgac

SEQ ID NO: 57 - DNA, 54 bp, Artificial Sequence
  Synthetic Construct
cacaatcccactcctttgtatatagccgacgccgacgccgccacctcgtgtctc

SEQ ID NO: 58 - DNA, 54 bp, Artificial Sequence; identical to SEQ ID NO: 57
  Synthetic Construct

SEQ ID NO: 59 - DNA, 54 bp, Artificial Sequence; identical to SEQ ID NO: 57
  Synthetic Construct

SEQ ID NO: 60 - DNA, 66 bp, Artificial Sequence
  Synthetic Construct
cctcgtgtctcatgacgcacaatcccactcctttgtatatagccgacgccgacgccgccgtatata

SEQ ID NO: 61-69 - skipped (9 entries)

SEQ ID NO: 70 - DNA, 40 bp, Artificial Sequence
  Synthetic Construct
atagcacaatgccgaccctttggccacgtggcaatatata

SEQ ID NO: 71 - DNA, 40 bp, Artificial Sequence
  Synthetic Construct
tcggcacaatgccgacgccacgtggcaagccgcctatata

SEQ ID NO: 72 - DNA, 70 bp, Artificial Sequence
  Synthetic Construct
ctcttgacgcacaatcccacttgtctctatatagccgccgcgggaaaagccgacgcgggaaaatatataa

SEQ ID NO: 73 - DNA, 73 bp, Artificial Sequence
  Synthetic Construct
gtaactgacgcacaatcccacttgtctctatatagccgccgcgggaaaagccgacgcgggaaaatatataagc

SEQ ID NO: 74 - DNA, 64 bp, Artificial Sequence
  Synthetic Construct
catccacaatcccactacctcgtgtctcgccgccgcgggaaaagccgacgcgggaaaatatata

SEQ ID NO: 75 - DNA, 55 bp, Artificial Sequence
  Synthetic Construct
acaccacaatacctcgtgtctcgccgacgcgggaaaggccgacgccgccgccgac

SEQ ID NO: 76 - DNA, 58 bp, Artificial Sequence
  Synthetic Construct
atgacacaatcccactcctttgtatatagccgacgccgacgccgccacctcgtgtctc

SEQ ID NO: 77 - DNA, 58 bp, Artificial Sequence
  Synthetic Construct
tctccacaatcccactcctttgtatatagccgacgccgacgccgccacctcgtgtctc

SEQ ID NO: 78 - DNA, 58 bp, Artificial Sequence
  Synthetic Construct
acgccacaatcccactcctttgtatatagccgacgccgacgccgccacctcgtgtctc

SEQ ID NO: 79 - DNA, 70 bp, Artificial Sequence
  Synthetic Construct
tttccctcgtgtctcatgacgcacaatcccactcctttgtatatagccgacgccgacgccgccgtatata

SEQ ID NO: 80-88 - skipped (9 entries)

SEQ ID NO: 89 - DNA, 25 bp, Artificial Sequence
  Synthetic Construct
tttaccgggaattgtcgaattaggc

SEQ ID NO: 90 - DNA, 16 bp, Artificial Sequence
  Synthetic Construct
gtgatctgaccgccgg

SEQ ID NO: 91 - DNA, 24 bp, Artificial Sequence
  Synthetic Construct
tttgccttggattttccacttgca

SEQ ID NO: 92 - DNA, 23 bp, Artificial Sequence
  Synthetic Construct
gagttggattcgtggccgcgcgg

SEQ ID NO: 93 - DNA, 23 bp, Artificial Sequence
  Synthetic Construct
tggtgggagatccatctgcgagg

SEQ ID NO: 94 - DNA, 25 bp, Artificial Sequence
  Synthetic Construct
tttctcatactcgttggctcgttgc

SEQ ID NO: 95 - DNA, 20 bp, Artificial Sequence
  Synthetic Construct
cccatcctccagtgctacgg

SEQ ID NO: 96 - DNA, 28 bp, Artificial Sequence
  Synthetic Construct
tttaggacctctagaggctacttctgcc

SEQ ID NO: 97 - DNA, 27 bp, Artificial Sequence
  Synthetic Construct
gcgaagcggcagagaatggcagggaaa

SEQ ID NO: 98 - DNA, 27 bp, Artificial Sequence
  Synthetic Construct
tttgataatagctatgaagattttgtg

SEQ ID NO: 99 - DNA, 50 bp, Artificial Sequence
  Synthetic Construct
cgagagagccgtctccctcttcgcttctcctctcccccccgtgatctgac

SEQ ID NO: 100 - DNA, 50 bp, Artificial Sequence
  Synthetic Construct
cgccggcgacgaccccctcccaccacccgccgccgccgccgcgcccccgc

SEQ ID NO: 101 - DNA, 50 bp, Artificial Sequence
  Synthetic Construct
cggcggcgtcgtcgtcttcctcctcttccccccgagttggattcgtggcc

SEQ ID NO: 102 - DNA, 50 bp, Artificial Sequence
  Synthetic Construct
cggcggatgggaggggaggagggaatggcggcggggaggaagaagcgggt

SEQ ID NO: 103 - DNA, 46 bp, Artificial Sequence
  Synthetic Construct
gatacaggtagagagaggtgagaaggcagtggtgggagatccatct

SEQ ID NO: 104 - DNA, 54 bp, Artificial Sequence
  Synthetic Construct
gcgaggtgcctccatggctgagaaggagggcaacctcgacgccgtcctcaagga

SEQ ID NO: 105 - DNA, 43 bp, Artificial Sequence
  Synthetic Construct
acctgccactcgcccactcctcctcctcccccatcctccagtg

SEQ ID NO: 106 - DNA, 51 bp, Artificial Sequence
  Synthetic Construct
ctacggtgtcacgcagccatggcgctccgcacgctggcctcgaggaaaacc

SEQ ID NO: 107 - DNA, 10 bp, Artificial Sequence
  Synthetic Construct
gccacgtggc

SEQ ID NO: 108 - DNA, 11 bp, Artificial Sequence
  Synthetic Construct
taagagccgcc

SEQ ID NO: 109 - DNA, 11 bp, Artificial Sequence
  Synthetic Construct
cctcgtgtctc

SEQ ID NO: 110 - DNA, 25 bp, Artificial Sequence
  Synthetic Construct
tttgttgtgtacttgatcgccgata

SEQ ID NO: 111 - DNA, 25 bp, Artificial Sequence
  Synthetic Construct
tttctcaggtggtgaattttgatcg

SEQ ID NO: 112 - DNA, 25 bp, Artificial Sequence
  Synthetic Construct
tttctatacgtatactacttccctc

SEQ ID NO: 113 - DNA, 25 bp, Artificial Sequence
  Synthetic Construct
tttccaaatcgccagattatcagta

SEQ ID NO: 114 - DNA, 25 bp, Artificial Sequence
  Synthetic Construct
tttctgaatgatttctaacgatcat

SEQ ID NO: 115 - DNA, 25 bp, Artificial Sequence
  Synthetic Construct
tttcattataattgggcacattgtg

SEQ ID NO: 116 - DNA, 25 bp, Artificial Sequence
  Synthetic Construct
tttctcggcccatctacgtgtcatg

SEQ ID NO: 117 - DNA, 25 bp, Artificial Sequence
  Synthetic Construct
tttaggcgaccacgtatctatctct

SEQ ID NO: 118 - DNA, 25 bp, Artificial Sequence
  Synthetic Construct
tttgcccaatcgcttcttcgaaacg

SEQ ID NO: 119 - DNA, 25 bp, Artificial Sequence
  Synthetic Construct
tttactcctagtcctctactgtttt

SEQ ID NO: 120 - DNA, 40 bp, Artificial Sequence
  Synthetic Construct
ctattatatattgccacgtggccaaagggtcggcattgtg

SEQ ID NO: 121 - DNA, 40 bp, Artificial Sequence
  Synthetic Construct
ccgatatataggcggcttgccacgtggcgtcggcattgtg

SEQ ID NO: 122 - DNA, 70 bp, Artificial Sequence
  Synthetic Construct
agagttatatattttcccgcgtcggcttttcccgcggcggctatatagagacaagtgggattgtgcgtca

SEQ ID NO: 123 - DNA, 73 bp, Artificial Sequence
  Synthetic Construct
ttacgcttatatattttcccgcgtcggcttttcccgcggcggctatatagagacaagtgggattgtgcgtcag

SEQ ID NO: 124 - DNA, 64 bp, Artificial Sequence
  Synthetic Construct
gatgtatatattttcccgcgtcggcttttcccgcggcggcgagacacgaggtagtgggattgtg

SEQ ID NO: 125 - DNA, 55 bp, Artificial Sequence
  Synthetic Construct
gtgtgtcggcggcggcgtcggcctttcccgcgtcggcgagacacgaggtattgtg

SEQ ID NO: 126 - DNA, 58 bp, Artificial Sequence
  Synthetic Construct
tcatgagacacgaggtggcggcgtcggcgtcggctatatacaaaggagtgggattgtg

SEQ ID NO: 127 - DNA, 58 bp, Artificial Sequence
  Synthetic Construct
gagagagacacgaggtggcggcgtcggcgtcggctatatacaaaggagtgggattgtg

SEQ ID NO: 128 - DNA, 58 bp, Artificial Sequence
  Synthetic Construct
gcgtgagacacgaggtggcggcgtcggcgtcggctatatacaaaggagtgggattgtg

SEQ ID NO: 129 - DNA, 70 bp, Artificial Sequence
  Synthetic Construct
gaaatatatacggcggcgtcggcgtcggctatatacaaaggagtgggattgtgcgtcatgagacacgagg